(19) United States
(12) Patent Application Publication
(10) Pub. No.: US 2022/0265730 A1
(43) Pub. Date: Aug. 25, 2022

(54) Engineered Bacteria Containing Biosensors for Precision Targeting and Containment

(71) Applicant: The Trustees of Columbia University in the City of New York, New York, NY (US)

(72) Inventors: Tal Danino, Brooklyn, NY (US); Tiffany CHIEN, Arcadia, CA (US); Tetsuhiro HARIMOTO, New York, NY (US)

(73) Assignee: The Trustees of Columbia University in the City of New York, New York, NY (US)

(21) Appl. No.: 17/737,562

(22) Filed: May 5, 2022

Related U.S. Application Data
(63) Continuation of application No. PCT/US2020/059035, filed on Nov. 5, 2020.
(60) Provisional application No. 62/930,665, filed on Nov. 5, 2019.

(51) Int. Cl. A61K 35/74; C12N 15/74; C12N 9/02; C12N 9/10; A61P 1/00; A61P 35/00

(52) U.S. Cl. CPC A61K 35/74; C12N 15/74; C12N 9/0008; C12Y 102/01011; A61K 2035/11; C12Y 206/01016; A61P 1/00; A61P 35/00; C12N 2830/002; C12N 9/1096

(57) ABSTRACT

The disclosure herein relates to engineered biosensor-containing bacteria, which is bacteria that contain at least one biosensor circuit, and uses thereof. A biosensor circuit can comprise an essential gene of the bacteria operably linked to an inducible promoter. Additionally, the bacteria can be engineered to be deficient in the endogenous copy of the at least one essential gene.

### CROSS REFERENCE TO RELATED APPLICATION

This application claims priority to U.S. Provisional Applications No. 62/930,665 filed on Nov. 5, 2019, both is incorporated by reference herein in its entirety.

### STATEMENT OF GOVERNMENTAL INTEREST

This invention was made with government support under LC160314, BC160541, W81XWH-17-1-0356 and W81XWH-17-1-0395 awarded by the Department of Defense and CA197649 and GM069811 awarded by the National Institutes of Health. The government has certain rights in the invention.

### BACKGROUND

With microbiome research demonstrating a prevalence of microorganisms within the human body, an emerging focus of synthetic biology is the engineering of microbes to supplant natural niches and act as living medicines (Riglar and Silver 2018; Ruder et al. 2011; Khalil et al. 2010; Kitada et al. 2018). Genetic programming of bacteria allows for sensing and responding to physiological conditions in situ and is posed to change paradigms for diagnosing and treating diseases such as inflammation, infection, and cancer (Riglar et al. 2017; Mao et al. 2018; Din et al. 2016; Danino et al. 2015). An essential element of this approach is precise control over microbial replication at disease sites, since uncontrolled bacterial growth can lead to severe side-effects including tissue damage and septic shock (Cao et al. 2019)). The engineering of genetic circuits to control bacterial growth at specific regions therefore provides an avenue for addressing this central challenge for clinical translation of next-generation microbial therapies.

The majority of bacterial therapies have relied on the natural tropism of bacteria in organs such as the gastrointestinal tract, skin, and tumors (Riglar et al. 2018; Danino et al. 2015; Nakatsuji et al. 2018). Many bacteria also have the ability to grow outside of their natural niches and quickly spread to unintended locations. Engineering approaches to controllably alter bacterial growth are being developed including the use of autotrophy for metabolites, essential genes, toxins, and dependency to synthetic amino acids (Lee et al. 2018; Mandell et al. 2015; Stirling et al. 2018; Chan et al. 2016). Coupling such systems with an environmentally-responsive biosensor can contain engineered bacteria from spreading to the surrounding environment (Piraner et al. 2017; Yu et al. 2012; Gallagher et al. 2015), but precise confinement of bacteria to specific organs has yet to be realized.

### SUMMARY

Disclosed herein are non-pathogenic bacteria comprising at least one essential gene under the control of an inducible promoter, which is a promoter which is regulated or induced in presence of certain factors

Inducible promoters allow regulation of gene expression and can be regulated by exogenously supplied compounds or agents, environmental or physiological actors such as temperature, or the presence of a specific physiological state, e.g., acute phase, a particular differentiation state of the cell, or in replicating cells only.

In some embodiments, the inducible promoter is induced by an external or exogenous agent.

In some embodiments, the promoter is a sensitive or responds to a particular environmental or physiological condition. This type of inducible promoter includes but is not limited to hypoxia-sensing promoters, pH sensing promoters, lactate sensing promoters and combinations thereof.

In some embodiments, the inducible promoter is induced by bacterial molecules, either from the bacteria themselves, e.g., autoinducers, or others. Examples of this type of inducible promoter includes but is not limited to quorum sensing promoters from genes including but not limited to LuxR, Las, Rhl, TraI/TraR, ExpI/ExpR-CarI/CarR, and Rpa.

The essential gene includes but is not limited to asd, glms acpP, dxr, ipxC, hemA, nadE, ribA, folA, pyrH, adk, tmk, gmk and glnS. Additionally, the non-pathogenic bacteria is deficient in the endogenous copy of the at least one essential gene.

In some embodiments, the bacteria further comprise additional or modified gene expression regulators chosen from the group including but not limited to antisense promoters, ribosome binding sites, origin of replication and protein degradation tags.

The inducible promoter can be on a plasmid or integrated into the genome of the bacteria. The plasmid can be low copy, medium copy or high copy.

In some embodiments, the bacteria can comprise more than one biosensor circuit, e.g., more than one different essential gene under the control of different inducible promoters. In some embodiments, the different inducible promoters are of the same type, e.g., the inducible promoters are all induced by a physiological condition. In some embodiments, the inducible promoters are of different types, e.g., one inducible promoter is induced by a physiological condition and another inducible promoter is induced by an autoinducer.

The bacteria may also include a therapeutic or diagnostic agent. The therapeutic agent or diagnostic agent can be encoded by a nucleic acid contained in a plasmid.

There are many uses of the engineered biosensor-containing bacteria, including but not limited to the treatment and diagnosis of cancer, gastrointestinal diseases and disorders, and skin diseases and disorders.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1—Schematic of biosensors for engineered bacteria tropism. FIG. 1 is a schematic of the engineered biosensors that sense specific oxygen, lactate and pH levels of organs that enable precise organotropism of bacteria in vivo. Different organs, ranging from liver (L), spleen (Sp), small intestine (S.I.), large intestine (L.I.) and tumor (T) exhibit varying biochemical signatures (Table 1). Bacteria programmed to sense pH and oxygen enhance bacteria colonization in the small and large intestine, respectively. Multiplexed lactate- and oxygen- AND logic gate sensing bacteria grow selectively within the tumor microenvironment.

FIG. 2—Design and characterization of hypoxia, lactate and pH biosensors. FIG. 2A shows the architecture of each sensor on the left. The architecture optimizes native bacteria promoters to sense specific environmental changes and express GFP. The hypoxia biosensor utilizes the pPepT promoter (or others) that relies on dimerized FNR to drive gene expression under low oxygen level. The lactate biosensor contains constitutive production of an LldR repressor, which de-represses the pLldR promoter in the presence of lactate. Lastly, the pH biosensor, based on the pCadC system, is regulated by the membrane-tethered transcriptional factor CadC. Biosensor strains were grown in a specified environment (0 and 20% oxygen, 0-10 mM lactate, and 5.5, 6, 6.5, 7 pH) for 16 hours. Fold change was calculated as fluorescent ratio of induced to un-induced state (n=3, mean±S.E.M). The right are graphs of the results showing bacteria that were grown in ±inducers (oxygen, 0-10 mM lactate and pH of 5.5-7) for 16 hours and fluorescence measured (n=3, mean±SEM). FIG. 2B-2D show induction data from lactate biosensor variants that were constructed by generating a library of plasmids containing a lactate promoter driving GFP and a constitutive promoter driving the lactate repressor. The plasmids were then co-transformed into E. coli Mach1 or an EcN strain, resulting in 24 generated variants. Induction data from 24 different strains with a high copy (FIG. 2B), middle copy (FIG. 2C) and low copy (FIG. 2D) reporter plasmid showing the level of GFP expression (a.u) at each external concentration of lactate (left graphs). The fold-change of the lactate biosensor was calculated as the ratio of GFP fluorescence in induced to non-induced states (right graphs). The level of repressor paired with the reporter is represented by the shades of color, with darker colors corresponding to increasing repressor copy numbers. The dotted (red) line in FIG. 2C represents the optimal strain that matched the criteria of low basal expression and high fold change upon induction. FIG. 2E shows the p end point GFP fluorescence of the in vitro characterization of pH biosensor pTC37 in 50 mM HEPES buffered media, grown in an incubator for 16 hours in pH 5.5-7 environment (n=3, ±S.E.M). FIG. 2F is a graph of hypoxia biosensor variants (pPepT, pVgb and FF+20) grown in normoxia to observe basal promoter expression utilizing GFP as proxy for 16 hours (n=3, ±S.E.M). pSodA promoter driving GFP expression was cultured in normoxia and hypoxia over 16 hours followed by fluorescence assay. GFP was normalized by pTac and fold change was calculated by GFP ratio between normoxia and hypoxia (n=2, ±S.E.M). FIG. 2G shows the three biosensors (pPepT, pTC1908, pTC37) chosen from the variants and each grown in pH of 5.3-7.3, 0, 0.1, 1, 10 mM of lactate and 0, 10, 20% oxygen levels for 16 hours to characterize their induction in intersecting environmental conditions. All strains were cultured and assayed for fluorescence signal compared to a control baseline condition with oxygen at 20%, lactate concentration at 0 mM and pH level at 7.3 (n=3, mean data, ±S.E.M=0.34, 0.32, 2.6 for pPepT, pTC1908 and pTC37).

FIG. 3—Engineering biosensor-dependent containment circuits and multiplexing for AND logic gate growth. FIG. 3A shows the design of modular containment circuit that includes a biosensor promoter (pPepT, pLldR, or pCadC) driving an essential gene (asd). To tune the sensitivity and reduce noise, additional regulators such as antisense promoters (pTac or pSodA), origin of replication (colE1, p15A, sc101 or genome integration), ribosomal binding site (RBS) or protein degradation tags (LAA) were utilized. FIG. 3B shows the characterization of biocontainment variants by escapee rate defined as the ratio between colonies grown in non-permissive conditions (normoxic, 0 mM lactate, and pH 7) and permissive conditions (hypoxic, 10 mM lactate, and pH 6). All variants were cultured for 12-16 hours and plated on LB agar plates with added supplements, after which colonies were counted the next day (n=3, mean±S.E.M). All variants drove an essential gene along with additional genetic parts such as anti-sense, tuned RBS strength, or degradation tag (LAA). Blue, red and green indicate hypoxia, lactate and pH driven containment circuit. The bottom panel shows the design of the circuit variants specifying changes in origin of replication, RBS, and regulators for each construct. FIGS. 3C-3E show the colony forming units (CFU)/ml of biosensor containment strain cultured in multiple physiological conditions versus wildtype. FIG. 3C shows hypoxia containment strain (pTH6-1) cultured under 0%, 10% or 20% oxygen. FIG. 3D shows lactate containment strain (pBK3-2/8) grown in 0, 0.1, 1 and 10 mM lactate. FIG. 3E shows pH containment strain (pTC085) grown in pH 5.2, 5.5, 6.0 and 7.0. All cultures were plated the after 16 hours on supplement-supplied agar plates followed by colony enumeration (n=3, ±S.E.M, LOD 200 CFU/ml). FIG. 3F is a graph of escapee rates of lactate-hypoxia AND logic gate circuit. The engineered bacteria were cultured in the different four conditions (none, 10 mM lactate supplemented, hypoxic condition and combination of 10 mM lactate with hypoxic condition). Escapee rate is calculated by ratio between non-permissive (no inducers, lactate only or hypoxic only) and permissive condition (both lactate and hypoxia). Two single circuits were grown in either lactate or hypoxic conditions (n=3, mean±S.E.M). Samples were grown for 16 hours and then plated on LB agar supplemented with DAP and D-glucosamine. Colonies were counted after incubating at 37° C. overnight. FIGS. 3G-3J are growth curve of the bacteria carrying lactate containment circuits at varying starting densities. Bacterial cultures were inoculated at starting densities 10⁶, 10⁵, 10⁴, and 10³ cells per well, all grown in culture±10 mM lactate. OD600 was measured every 15 minutes for 18 hours on a plate reader. (n=3, ±S.E.M). FIG. 3G shows starting density of 10⁶ cells per well. FIG. 3H shows starting density of 10⁵ cells per well. FIG. 3I shows starting density of 10⁴ cells per well. FIG. 3J shows starting density of 10³ cells per well. FIG. 3K is a schematic of lactate-hypoxia AND logic gate circuit that consist of both the hypoxia promoter pPepT driving essential gene asd and lactate biosensor pLldR driving a second essential gene glms. FIG. 3L is a graph of the growth dynamics of lactate-hypoxia containment strain in four different conditions (none, 10 mM lactate supplemented, hypoxia condition, and combination of 10 mM lactate with hypoxic condition). Samples were collected every 3 hours and plated on LB agar supplemented with DAP and D-glucosamine (n=3, ±S.E.M). Colonies were counted after 37° C. incubation for 16 hours.

FIG. 4—Computational modeling of biosensors. Biosensors are modeled under regulation of transcription activators (FNR, CadC) or repressors (LldR) with varying environmental conditions. FIG. 4A shows the hypoxia biosensor. FIG. 4B shows the lactate biosensor. FIG. 4C shows the pH biosensor. The biosensor circuit schematics as shown in FIG. 2 is shown on the left with modeling in silico predictions (middle panel) compared to in vitro experimental results (right panel) (n=3, ±S.E.M). Lactate and pH biosensor in vitro GFP fluorescent result is normalized by OD. Hypoxia biosensor in vitro GFP fluorescence was measured in an anaerobic chamber and are normalized by data from constitutive promoter pTac. FIGS. 4D-4F are heatmaps showing bacterial growth in two-dimensional space with lactate concentration of 0, 0.1, 1 and 10 mM, pH levels of 5.3, 5.8, 6.3 and 7.3, and oxygen levels of 0, 10 and 20% after 2 hours. FIG. 4D shows 1-input single containment strains grow in response to hypoxia (blue), high lactate (red) and low pH (green) environment. FIG. 4E shows 2-input AND gate containment strains. Bacterial growth is further restricted to where two specified environmental conditions overlap. For lactate hypoxia AND gate containment strain (purple), maximum growth is observed at lactate concentration 10 mM and oxygen level of 0%. For pH hypoxia AND gate containment strain (brown), maximum growth is seen at pH of 5.3 and oxygen level of 0% Lastly, for lactate pH AND gate containment strain (orange), maximum bacterial growth is seen at pH of 5.3-5.8 and lactate of 10 mM, regardless of oxygen content. FIG. 4F shows the combination of all three biosensors (black), bacterial growth is limited to when pH is 5.3-5.8, lactate of 10 mM and oxygen level of 0%. FIG. 4G is a graph of computational modeling of lactate hypoxia AND gate containment strain growth over time in permissive (high lactate and hypoxia) and non-permissive (none, high lactate or hypoxia only) conditions. FIG. 4H is a graph of growth simulation of pH hypoxia AND gate containment strain in permissive (low pH and hypoxia) and non-permissive (none, low pH or hypoxia only) conditions. FIG. 4I is a graph of growth simulation of lactate pH AND gate containment strain in permissive (high lactate and low pH) and non-permissive (none, high lactate or low pH only) conditions. FIGS. 4J-4L show the computational simulation of single and AND gate containment strain growth kinetics. FIG. 4J shows pH containment strain. FIG. 4K shows lactate containment strain. FIG. 4L shows hypoxia containment strain. All reach stationary stage sooner compared to hypoxia lactate AND gate containment strain (purple), pH lactate AND gate containment strain (orange) or hypoxia pH AND gate containment strain (brown) in silico. Parameters set for growth corresponds to pH 5, lactate concentration of 10 mM and 0% oxygen level.

FIG. 5—Engineered biosensors respond to physiological cues in vitro. FIG. 5A shows the cell culture media supernatant from four cancer cell lines (A20, CT26, 368T1 and 4T1) were collected twice a day over five days and then cultured with the three biosensors (pTC1908, pTC37 and pPepT). Levels of lactate and pH of A20, CT26, 368T1 and 4T1 cell supernatants were measured over 5 days. FIG. 5B is a graph of lactate concentration of the 4 different cancer cell lines supernatant over time. All four cell lines were cultured under the same conditions. Lactate concentration of the collected supernatants exhibited increasing trend with all cell lines starting with lactate levels less than 5 mM. As time progressed 368T1 and 4T1 cell supernatant exhibited more lactate accumulation compared to the other two cell lines. FIG. 5C is a graph of pH concentration of the 4 different cancer cell lines supernatant over time. All four cell lines were cultured under the same conditions. The pH level from all cell line supernatants showed decreasing trends, as 368T1 and 4T1 exhibited a sharper decrease in pH compared to A20 and CT26 cells. FIG. 5D shows fluorescence activation of lactate biosensors when cultured in the collected supernatant overnight at 37° C. FIG. 5E shows fluorescence activation of pH biosensors when cultured in the collected supernatant overnight at 37° C. FIG. 5F shows lactate concentration from 6 different lung cancer cell lines and 1 lung fibroblast cell line supernatant exhibiting increasing trend. FIG. 5G shows lactate biosensor bacteria cultured with mammalian cell supernatants (6 lung cancer cell lines and a human lung fibroblast) for 16 hours, followed by fluorescence assay. Elevated GFP fluorescence was observed in response to increased lactate concentrations (n=3, ±S.E.M). FIG. 5H shows fluorescence activation of hypoxia biosensors when cultured in the collected supernatant overnight at 37° C. Hypoxia biosensor cultured in cell media supernatant was grown in ±oxygen conditions. GFP signal from hypoxia biosensor was normalized by constitutive promoter control. (n=3, mean±S.E.M).

FIG. 6—Engineered biosensors enable redirected growth tropism and biocontainment in mouse gut. FIG. 6A is a schematic of the experiment. Mice were orally administered bacteria. Fresh fecal pellets were collected every day for 7 days, homogenized and plated on LB agar plates with antibiotic (Abx) selection±DAP. Mice were sacrificed at the end of the experiment (day 7), and the gastrointestinal tract was sectioned into 5 regions (S1, upper small intestine track; S2, lower small intestine track; C, caecum; L1, upper large intestine track; L2, lower large intestine track). The regions were homogenized and plated on LB agar plates with antibiotic selection and DAP. Colonies were counted the following day. FIG. 6B is a graph of colony counts from fresh fecal pellet homogenates recovered by plating in permissive and non-permissive conditions. (Nislux: n=7, pH (pTC085): n=7, hypoxia (pTH6-1): n=8 per time point, mean±S.E.M. ***p=0.0005 two-way analysis of variance (ANOVA) with Tukey's multiple comparisons test, limit of detection (LOD) 10³ CFU/g). FIG. 6C shows the individual bacterial distribution of positive control E. coli Nissle 1917 with luxCDABE (Nislux) demonstrated by recovered CFU/g from small intestine 1 (S1), small intestine 2 (S2), caecum (C), large intestine 1 (L1) and large intestine 2 (L2). LOD, 10³ CFU/g. FIG. 6D shows the individual bacterial distribution of hypoxia containment strain pTH6-1 demonstrated by recovered CFU/g from small intestine 1 (S1), small intestine 2 (S2), caecum (C), large intestine 1 (L1) and large intestine 2 (L2). LOD, 10³ CFU/g. FIG. 6E shows the individual bacterial distribution of pH containment strain pTC85 demonstrated by recovered CFU/g from small intestine 1 (S1), small intestine 2 (S2), caecum (C), large intestine 1 (L1) and large intestine 2 (L2). LOD, 10³ CFU/g. FIG. 6F is a graph of fold change of bacteria relative to S 1. CFU/g from each location scaled by respective CFU/g from S 1. (Nislux: n=13, pH (pTC085): n=8, hypoxia (pTH6-1): n=15, mean±S.E.M, *p=0.0152 two-way analysis of variance (ANOVA) with Tukey's multiple comparisons test). FIG. 6G shows the fold change of bacteria relative to S1 and Nislux. CFU/g from each location scaled by respective S1 as well as Nislux. Dotted line indicate wildtype base line. (Nislux: n=13, pH (pTC085): n=8, hypoxia (pTH6-1): n=15, mean±S.E.M. ***p=0.0006 two-way analysis of variance (ANOVA) with Tukey's multiple comparisons test). FIG. 6H is a graph of CFU measurements experimentally measured at 5 sections of the gut (S1, S2, C, L1 and L2) from a mouse colonized with wildtype bacteria (Nislux). Y-scale logarithmic fit from point to point was applied to produce wildtype CFU data at each hundredth segment in the gut from S1 to L2. FIG. 6I shows the computational modeling of the hypoxia containment strain used to predict CFU levels at each segment of the gut. Nislux CFU of each gut compartment was set as carrying capacity for computational prediction (i.e., Nmax at L1=1010, as shown in FIG. 6H). Since increasing con-centration of essential gene products resulted in faster cellular growth, growth rate was set proportional to the product of essential gene expression in the simulation. FIG. 6J shows the computational modeling of the pH containment strain used to predict CFU levels at each segment of the gut. Nislux CFU of each gut compartment was set as carrying capacity for computational prediction (i.e., Nmax at L1=1010, as shown in FIG. 6H.) Since increasing concentration of essential gene products resulted in faster cellular growth, growth rate was set proportional to the product of essential gene expression in the simulation. FIG. 6K shows input pH obtained using a second-degree polynomial fit of the means of measured pH data (top) and input hypoxia obtained using a second-degree polynomial fit of hypoxia data found in literature (bottom). FIG. 6L is a graph of CFU along the gut for Nislux, pH and hypoxia were each divided by their S1 CFU value to visualize bacterial distribution along the gut. FIG. 6M is a graph of the computation model pH and hypoxia CFU/S1 values were normalized using the wildtype Nislux CFU/S1 values.

FIG. 7—Multiplexed biosensor achieves enhanced specificity of bacteria tumor colonization. FIG. 7A is a schematic of the engineered bacteria biosensors were co-cultured in tumor spheroids and monitored for biosensor activation. FIG. 7B is a graph of biosensors (pPepT, pTC1908 and pTC037) transformed and characterized in Salmonella Typhimurium ELH1301 strain. Bacteria were grown in a specified environment for 16 hours, followed by fluorescence assay. Fold change calculated from GFP of induced (0% oxygen, 10 mM lactate and pH 5.8) over GFP of un-induced condition (20% oxygen, 0 mM lactate and neutral pH). FIGS. 7C-7E are representative images of biosensors in tumor spheroids. (Scale bar, 200 μm) (top) and corresponding space-time diagram demonstrating radially averaged fluorescence intensity (bottom). FIG. 7C shows lactate (pTC1908) biosensor. FIG. 7D shows pH (pTC037) biosensor. FIG. 7E shows hypoxia (pPepT) biosensor. FIG. 7F shows a time series images of spheroids cocultured with S. typhimurium carrying plasmid expressing constitutive GFP (scale bar=200 μm). FIG. 7G is a graph of the average GFP fluorescence signal from spheroids over 9 days (n=3, ±S.E.M). FIG. 7H shows replicates of tumor spheroids colonized by lactate biosensor bacterial strains where promoter activation was measured by automated spatiotemporal image analysis. Average florescence signal of each biosensor colonized spheroids was also tracked over 8-14 days (n=3, ±S.E.M). FIG. 7I shows replicates of tumor spheroids colonized by pH biosensor bacterial strains where promoter activation was measured by automated spatiotemporal image analysis. Average florescence signal of each biosensor colonized spheroids was also tracked over 8-14 days (n=3, ±S.E.M). FIG. 7J shows replicates of tumor spheroids colonized by hypoxia biosensor bacterial strains where promoter activation was measured by automated spatiotemporal image analysis. Average florescence signal of each biosensor colonized spheroids was also tracked over 8-14 days (n=3, ±S.E.M). FIG. 7K shows the recovered colony counts of wildtype S. typhimurium ELH1301, ELH1301 ΔasdΔglms and hypoxia-lactate circuit tested in spheroid platform. All strains were co-cultured in spheroids for 6 days followed by homogenizing the spheroid and plating them on LB agar plates. (n=3, mean±S.E.M, LOD 20 CFU). FIG. 7L is a graph of CFU of S. typhimurium ELH1301 lacking essential genes (ΔasdΔglms) transformed with lactate-hypoxia circuit driving both asd and glms genes, hypoxia circuit driving asd gene only, and lactate circuit driving glms gene only. Bacteria were co-cultured with tumor spheroids for 6 days, followed by dissociation of tumor spheroids and plating on agar plates for CFU enumeration (n=4, ±S.E.M, LOD 20 CFU). FIG. 7M is a schematic of BALB/c mice (n=5 per group) were implanted subcutaneously with 5×10⁶ CT26 cells on one hind flank. When tumor volumes were 100-150 mm³, mice were intravenously administered ELH1301 (WT), double knockout (ΔasdΔglms) or lactate-hypoxia circuit (in (ΔasdΔglms). After 2 days, tumor, liver and spleen were homogenized and plated on LB agar plates with supplements. Bacteria colonizing tumor (G), spleen (H) and liver (I) tissues were quantified and counted after 1 day. (****p<0.0001, **p=0.0011 one-way analysis of variance (ANOVA) with Bonferroni's multiple comparisons test, n=5, mean±S.E.M, tumor and spleen LOD 103 CFU/g, liver LOD 102 CFU/g). FIG. 7N is a graph of number of bacteria colonizing in the tumor. FIG. 7O is a graph of the number of bacteria colonizing in the spleen. FIG. 7P is a graph of the number of bacteria colonizing in the liver. FIG. 7Q shows the ratio of tumor:spleen of bacterial CFU/g calculated from extracted organs. Ratio of engineered strain is >100 fold higher than non-engineered strain (****p<0.0001 one-way analysis of variance (ANOVA) with Bonferroni's multiple comparisons test, n=5, ±S.E.M). FIG. 7R shows the ratio of tumor:liver of bacterial CFU/g calculated from extracted organs. Ratio of engineered strain is >100 fold higher than non-engineered strain (****p<0.0001 one-way analysis of variance (ANOVA) with Bonferroni's multiple comparisons test, n=5, ±S.E.M). FIG. 7S shows the resulting CFU/g of 104 CFU engineered strain cultured for 24 hours in homogenized tissue only (non-permissive), homogenized tissue with 10 mM lactate and 0% oxygen (permissive) or homogenized tissue with supplements (rescue), and plated on agar plates. Colonies were counted after 16 hours (n=3, mean, ±S.E.M, LOD 20 CFU/g).

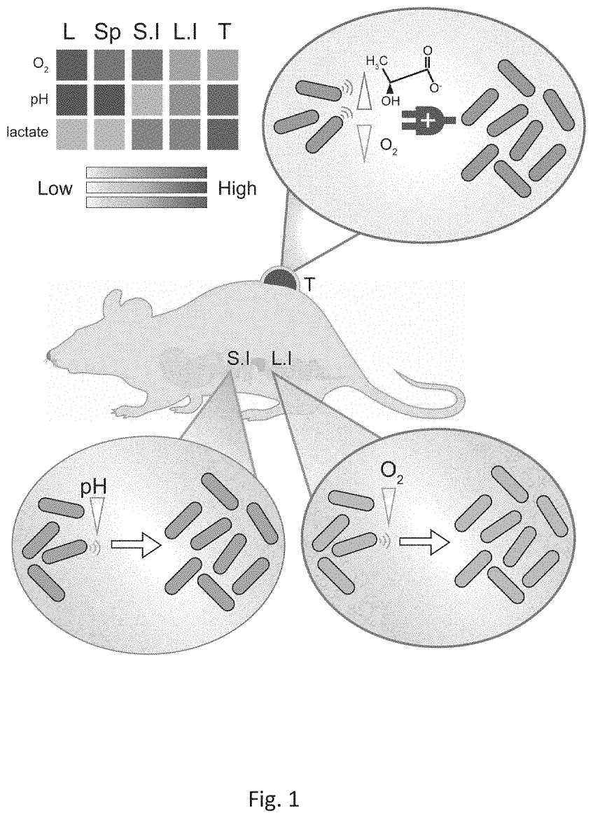

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The compositions and methods disclosed herein are particularly desirable for human medical applications.

The term “patient” as used in this application means a human subject.

The term “agent” as used herein means a substance that produces or is capable of producing an effect and would include, but is not limited to, chemicals, pharmaceuticals, biologics, small organic molecules, antibodies, nucleic acids, peptides, and proteins.

As used herein “non-pathogenic bacteria” refer to bacteria that are not capable of causing disease or harmful responses in a host. In some embodiments, non-pathogenic bacteria are gram-negative bacteria. In some embodiments, non-pathogenic bacteria are gram-positive bacteria. In some embodiments, non-pathogenic bacteria do not contain lipopolysaccharides (LPS). In some embodiments, non-pathogenic bacteria are commensal bacteria. Examples of non-pathogenic bacteria include, but are not limited to certain strains belonging to the genus Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli Nissle, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii. Naturally pathogenic bacteria may be genetically engineered to provide reduce or eliminate pathogenicity.

The term “probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. In some embodiments, the probiotic bacteria are gram-negative bacteria. In some embodiments, the probiotic bacteria are gram-positive bacteria. Some species, strains, and/or subtypes of non-pathogenic bacteria are currently recognized as probiotic bacteria. Examples of probiotic bacteria include but are not limited to certain strains belonging to the genus Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii. The probiotic may be a variant or a mutant strain of bacterium. Non-pathogenic bacteria may be genetically engineered to enhance or improve desired biological properties, e.g., survivability. Non-pathogenic bacteria may be genetically engineered to provide probiotic properties. Probiotic bacteria may be genetically engineered or programmed to enhance or improve probiotic properties.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “plasmid” means the vehicle by which a DNA or RNA sequence (e.g., a foreign gene) can be introduced into a host cell, so as to transform the host and promote expression (e.g. transcription and translation) of the introduced sequence. Plasmids typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A plasmid is generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. Programmable plasmids will often include one or more replication systems for cloning or expression, one or more markers for selection in the host, e.g. antibiotic resistance, and one or more expression cassettes.

As used herein a “pharmaceutical composition” refers to a preparation of engineered biosensor-containing bacteria of any of the foregoing embodiments with other components such as a pharmaceutically acceptable suitable carrier and/or excipient.

The term “pharmaceutically-acceptable” refers to molecular entities and compositions that do not produce an allergic or similar untoward reaction when administered to a host, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

As used herein, the term “carrier” refers to a diluent, adjuvant, excipient, or vehicle with which the therapeutic is administered, and includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art.

The term “excipient” refers to an inert substance added to a pharmaceutical composition to further facilitate administration of an active ingredient. Examples include, but are not limited to, calcium bicarbonate, calcium phosphate, various sugars and types of starch, cellulose derivatives, gelatin, vegetable oils, polyethylene glycols, and surfactants, including, for example, polysorbate 20.

The term “tropism” as used herein indicates the growth and movement of organisms toward environmental stimulus. For example, tissue tropism describes the tissues of a host that supports growth of particular virus or bacteria. The term “bacteria tropism” as defined herein is the ability of bacteria to grow in response to specified environmental features (i.e., low pH, high lactate and hypoxia) that correspond to tissue signatures.

The articles “a” and “an,” as used herein, should be understood to mean “at least one,” unless clearly indicated to the contrary.

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Programmable DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Disclosed herein are bacteria engineered to contain biosensors that sense and respond to multiple physiological conditions which can be used target and grow the bacteria in specific organs or tumors within the body. As shown herein, by tuning and multiplexing the biosensors responsive to physiological cues as well as other inducers including exogenous compounds or agents and bacterial molecules such as autoinducers, precision targeting within organs and tumors can be achieved. This approach will enhance the specificity, effectiveness and safety of bacterial based therapeutics and diagnostics.

The critical design of the biosensor circuit is the coupling of bacterial growth with inducible promoters including physiological or environmental sensitive or responsive promoters. While inducible promoters have been used widely to highly express genes of interest with low basal expression, many natural promoters possess relatively low fold dynamic ranges (Chen et al. 2018; Wan et al. 2019). This lack of promoter activity has been a challenge for in vivo microbial applications since insufficient expression or off-target leakiness leads to lack of efficacy and safety, respectively. In this study, engineered promoter-biosensor machinery was coupled with bacterial growth via expression of essential genes. Because of the low gene expression needed for essential genes, minimal promoter activity was sufficient to allow bacterial growth at specified environmental conditions. This signal amplification through replication successfully achieved greater than 1000-fold differences in bacterial number using three independent biosensors. In terms of specificity, the ideal promoter would possess threshold-like activation in the desired environmental condition compared to a control. However, most natural and engineered promoters display a more graded response, observed in the biosensor variants. Nonetheless, amplification of the promoter signal through coupling to essential genes produces an improved profile due to the non-linear amplification of exponential growth. As a result, oral delivery of the probiotics with these biocontainment circuits demonstrated shifts in bacterial in accordance with oxygen and pH levels along the murine gut.

While the amplification through a single biosensor-growth coupling results in enhanced tropism, the system was subsequently engineered to incorporate multiple biosensor circuits to further enhance tropism. By using orthogonal biosensor-essential gene pairs, bacterial growth can be regulated with multiple environmental signatures or other regulators or inducers in an AND logic gate manner. This is particularly important for systemic bacterial delivery where microbes have access to multiple organs and can induce severe toxicity upon off-target colonization. As a proof-of-concept, an improvement in bacterial tumor specificity was shown by multiplexing oxygen and lactate biosensors. Although similar oxygen or lactate conditions can individually be found in other organs, the use of two environmental signatures increased the specificity to the tumor, resulting in 100-fold reduction in off-target colonization.

Utilizing a computational model, it was further demonstrated that coupling of multiple biosensors can indeed lead to improved tropism in relevant physiological range.

The approach described here can enable future precision targeting of specific physiological regions. For example, the use of the biosensors can be used to control the expression of therapeutic payloads to enhance efficacy and avoid off-target toxicity. More precise activation using multiplexed biosensors can be used to differentiate microenvironment of various tumor types, potentiating the use of bacteria as precision diagnostic devices. In addition, this approach can be generalized to other bacterial species and targets for biomedical research and translation to human disease. As translation of engineered microbes for various technological applications continues, robust and precise engineering of bacterial localization to desired environments will provide a useful method to improve biocontainment and safety to target specific sites for local delivery of treatment options.

The engineered biosensor-containing bacteria described herein comprise at least non-pathogenic bacteria and at least one biosensor circuit contained on a plasmid or genomically integrated. In some embodiments, the engineered biosensor-containing bacteria comprise more than one biosensor circuit. In some embodiments, the engineered biosensor-containing bacteria also comprise one or more additional plasmids. The one or more plasmids may produce a therapeutic agent, i.e., one or more plasmids comprising a nucleic acid sequence which encodes a therapeutic agent, or may produce a diagnostic agent, i.e., one or more plasmids comprising a nucleic acid sequence which encodes a diagnostic agent. In some embodiments, the engineered biosensor-containing bacteria also comprise additional components.

Bacteria

In some embodiments, the bacteria are obligate anaerobic bacteria. In some embodiments, the bacteria are facultative anaerobic bacteria. In some embodiments, the bacteria are aerobic bacteria. In some embodiments, the bacteria are gram-positive bacteria. In some embodiments, the bacteria are gram-negative bacteria. In some embodiments, the bacteria are non-pathogenic bacteria. In some embodiments, the bacteria are commensal bacteria. In some embodiments, the bacteria are probiotic bacteria. In some embodiments, the bacteria are naturally pathogenic bacteria that are modified or mutated to reduce or eliminate pathogenicity. Exemplary bacteria include, but are not limited to, Bacillus, Bacteroides, Bifidobacterium, Brevibacteria, Caulobacter, Clostridium, Enterococcus, Escherichia coli, Lactobacillus, Lactococcus, Listeria, Mycobacterium, Saccharomyces, Salmonella, Staphylococcus, Streptococcus, Vibrio, Bacillus coagulans, Bacillus subtilis, Bacteroides fragilis, Bacteroides subtilis, Bacteroides thetaiotaomicron, Bifidobacterium adolescentis, Bifidobacterium bifidum, Bifidobacterium breve UCC2003, Bifidobacterium infantis, Bifidobacterium lactis, Bifidobacterium longum, Clostridium acetobutylicum, Clostridium butyricum, Clostridium butyricum M-55, Clostridium butyricum miyairi, Clostridium cochlearum, Clostridium felsineum, Clostridium histolyticum, Clostridium multifermentans, Clostridium novyi-NT, Clostridium paraputrificum, Clostridium pasteureanum, Clostridium pectinovorum, Clostridium perfringens, Clostridium roseum, Clostridium sporogenes, Clostridium tertium, Clostridium tetani, Clostridium tyrobutyricum, Corynebacterium parvum, Escherichia coli MG1655, Escherichia coli Nissle 1917, Listeria monocytogenes, Mycobacterium bovis, Salmonella choleraesuis, Salmonella typhimurium, and Vibrio cholera.

In some embodiments, the bacteria are a non-pathogenic E. coli. In some embodiments, the bacteria are the probiotic bacteria, E. coli Nissle 1917 bacteria.

In some embodiments, the bacteria are Salmonella typhimurium.

In some embodiments, the bacteria without further manipulation or engineering are capable of activating the innate immune system via intracellular receptors and pathways including STING leading to type I IFN production, dendritic cell priming and T cell activation and thus are naturally occurring therapeutic agents.

In some embodiments, the bacteria without further manipulation or engineering are beneficial to the digestive and/or the immune system. These bacteria include but are not limited to Bifidobacterium, Escherichia coli, and Lactobacillus.

In some embodiments, essential genes are deleted from the bacteria such that these genes can be put under the control of an inducible promoter. These essential genes include but are not limited to asd, glms acpP, dxr, ipxC, hemA, nadE, ribA, folA, pyrH, adk, tmk, gmk and glnS. Methods for deleting these genes from bacteria are known in the art.

Biosensor Circuits

The bacteria disclosed herein comprise at least one biosensor circuit which can be an essential gene of the bacteria operably linked to an inducible promoter.

These essential genes include but are not limited to asd, glms acpP, dxr, ipxC, hemA, nadE, ribA, folA, pyrH, adk, tmk, gmk and glnS.

Inducible promoters allow regulation of gene expression and can be regulated by exogenously supplied agents or compounds, environmental or physiological factors such as temperature, or the presence of a specific physiological state, e.g., acute phase, a particular differentiation state of the cell, or in replicating cells only.

In some embodiments, the inducible promoter is regulated or induced by an external or exogenous compound or agent.

Examples of inducible promoters regulated by exogenous agents include but are not limited to a zinc-inducible sheep metallothionine (MT) promoter, a dexamethasone (Dex)-inducible mouse mammary tumor virus (MMTV) promoter, a T7 polymerase promoter system, a ecdysone insect promoter, a tetracycline-repressible system, a tetracycline-inducible system, a RU486-inducible system, and a rapamycin-inducible system.

In some embodiments, the inducible promoter is sensitive or responsive to a particular physiological or environmental condition. Disclosed herein are biosensor circuits capable of distinguishing oxygen, lactate and pH, which are physiological indicators found throughout the body as well as tumors.

Disclosed herein is the use of a hypoxia-sensing promoter. In hypoxia-sensing circuits, oxygen binds to the fumarate and nitrate reductase (FNR) monomers at physiological oxygen levels. Under hypoxic conditions, no oxygen is available for FNR binding, which results in dimerization and activation of FNR regulated promoters. Inducible bacterial promoters including but not limited to FF+20, pVgb, pPepT and Pvgb, which sense 0.5-5% oxygen in the tumor environmental range can be used in these circuits. In other embodiments, alternative oxygen dependent promoters can be used including but not limited to pPflE and pFnrS.

One lactate sensor circuit described herein was derived from a LldPRD operon and was constructed on two plasmids—a lactate-inducible reporter plasmid driving expression of a gene of interest, and a repressor plasmid, which produces LldR to inhibit the reporter gene unless lactate is present to bind and deactivate repressor dimers. Other lactate sensor circuits can be engineered by pairing different copy numbers of the gene expression plasmid and repressor plasmid to achieve varying dynamic ranges.

One pH sensor circuit described herein used a pCadC promoter regulated by a membrane tethered activator protein (CadC), which has increased activity in acid environments. Other pH sensor circuits can be engineered by using pydeP promoter that is regulated by activator protein EvgS. pSpeF pAsr, and pAdiA can also be used as pH sensor.

Circuits which are sensitive to other physiological indicators in the body and tumors include but are not limited to limited to temperature, glucose, propionate, butyrate sensors and tetrathionate sensors.

In some embodiments, the inducible promoter is induced by bacterial molecules, either from the bacteria themselves, i.e., autoinducers, or others. Examples of this type of inducible promoter includes but is not limited to quorum sensing promoters from genes including but not limited to LuxR, Las, Rhl, TraI/TraR, ExpI/ExpR-CarI/CarR, and Rpa

In order to put the bacterial replication and growth under the control of one or more biosensor circuits, an essential gene of the bacteria must be under control of the biosensor promoter. Exemplified herein was the essential gene asd, involved in lysine, threonine, and methionine biosynthesis and can be rescued by diaminopimelic acid (DAP). Another essential gene is glms, which encodes for glucosamine-6-phosphate synthase which can be rescued by D-glucosamine. Other essential genes can be determined by one of skill in the art depending on the bacteria used for the engineered biosensor-containing bacteria. In some embodiments, the essential gene must be knocked out or otherwise made inactive in the genome of the bacteria.

The biosensor circuit includes at least one inducible promoter and an essential gene of the bacteria under control of the inducible promoter. However, the constructions of biosensor circuits that are highly sensitive and specific to physiological as well as other indicators can require additional components. These various gene expression regulators, i.e, activators and repressors, include but are not limited to antisense promoters including but not limited to pTac and pSodA, ribosome binding sites, and protein-degradation tags. Increasing or decreasing these gene expression regulators allows fine tuning of the circuits. In other words, by varying the gene expression regulators, the response of the circuit can be more precise and targeted to a particular level of the physiological indicator. Methods for determining the necessary gene expression regulators and adding them to the biosensor circuits are known in the art.

In particular, increasing or decreasing ribosome binding sites (RBS) downstream from the promoter results in increased precision of the biosensor circuit. As shown, decreasing ribosome binding strength increased the sensitivity of the pH biosensor circuit. Methods for increasing or decreasing ribosome binding sites are known in the art.

The biosensor circuits can be contained on a plasmid or integrated into the genome of the bacteria. Examples of plasmid include but are not limited to pColE1, p15A, pAH162, and pSC101. In some embodiments, the plasmid is high copy. In some embodiments, the plasmid is medium copy. In some embodiments, the plasmid is low copy. Again varying the copy number of the plasmids encoding the biosensor circuit promoters, variants of the engineered biosensor-containing bacteria can be fine-tuned to particular physiological conditions in particular tumors and organs.

Additionally, varying the origin of replication (colE1, p15a or sc101 origin of replication or single genome integration) also allows for increased sensitivity and specificity of the biosensor circuit. Methods for varying the origin of replication are known in the art.

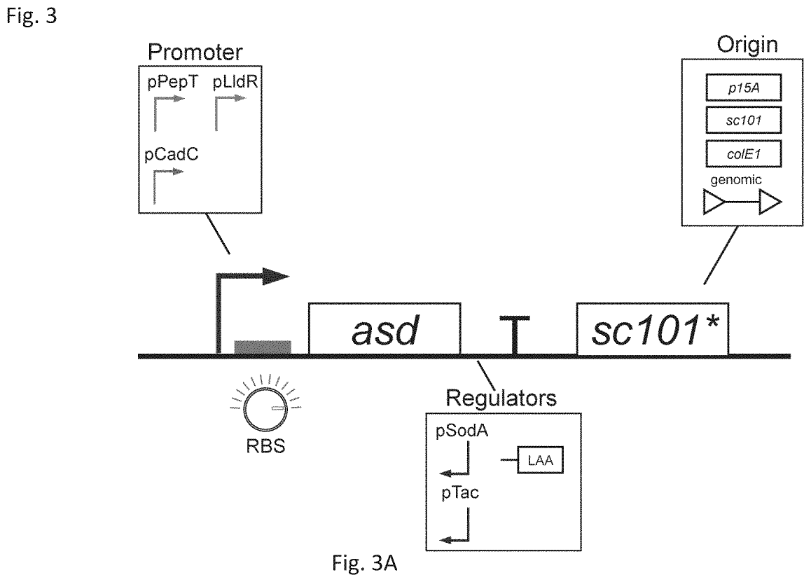

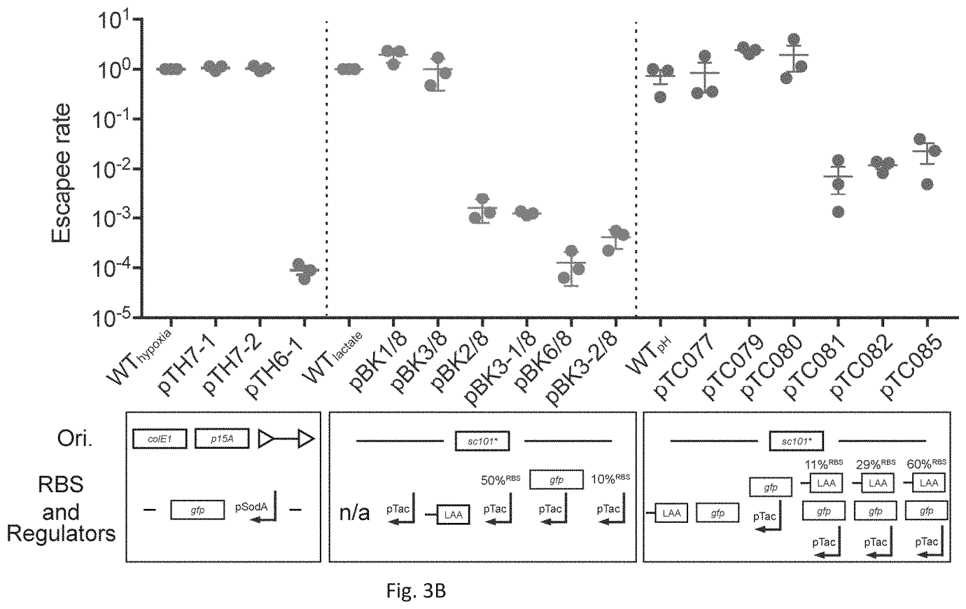

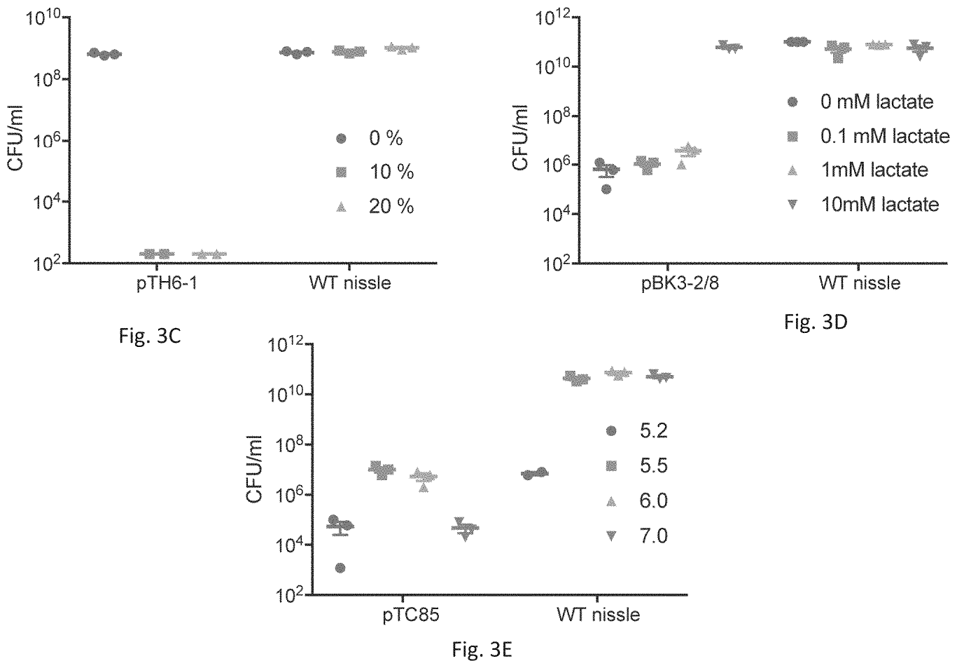

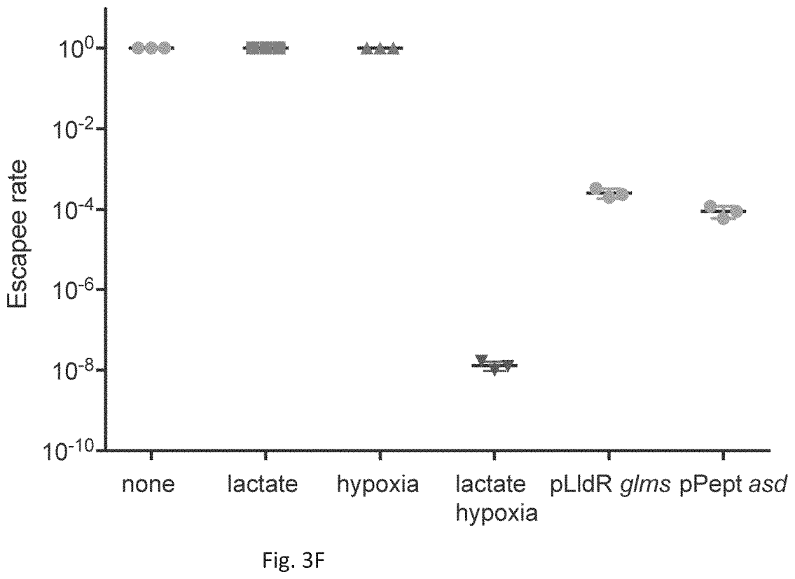

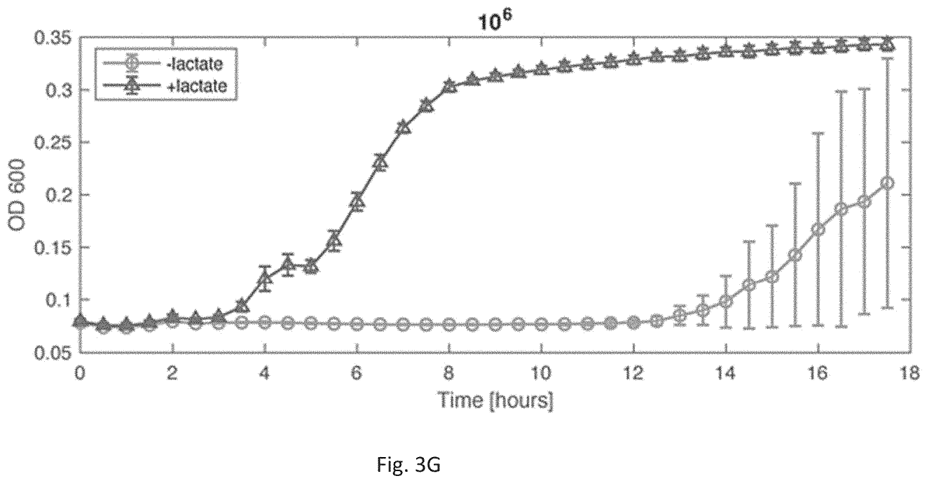

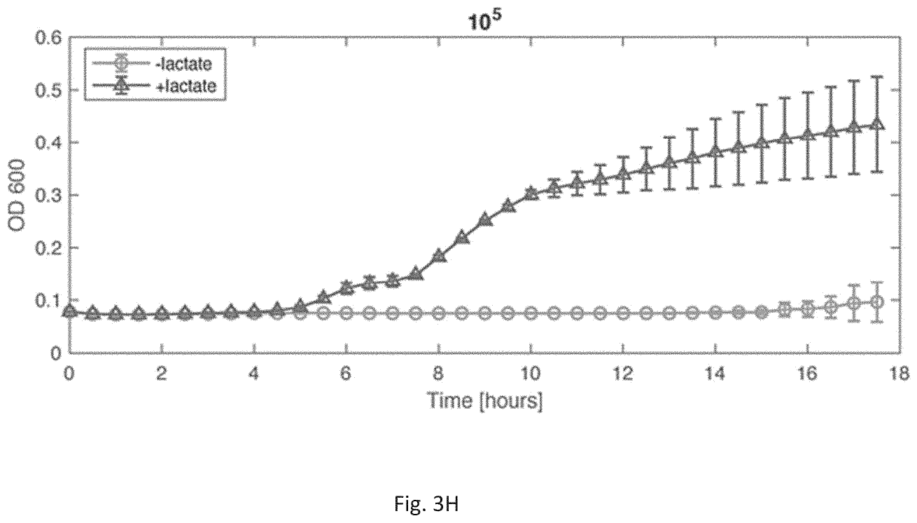

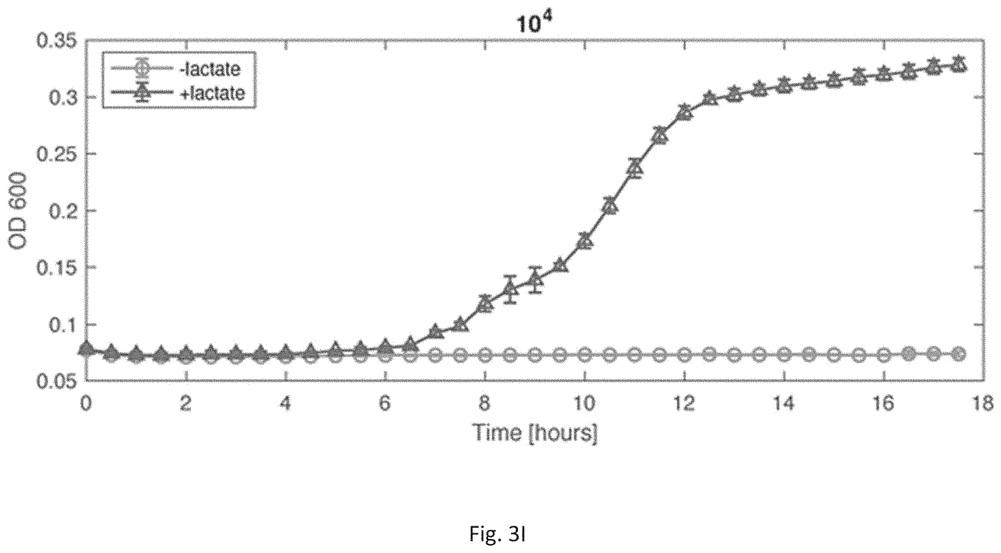

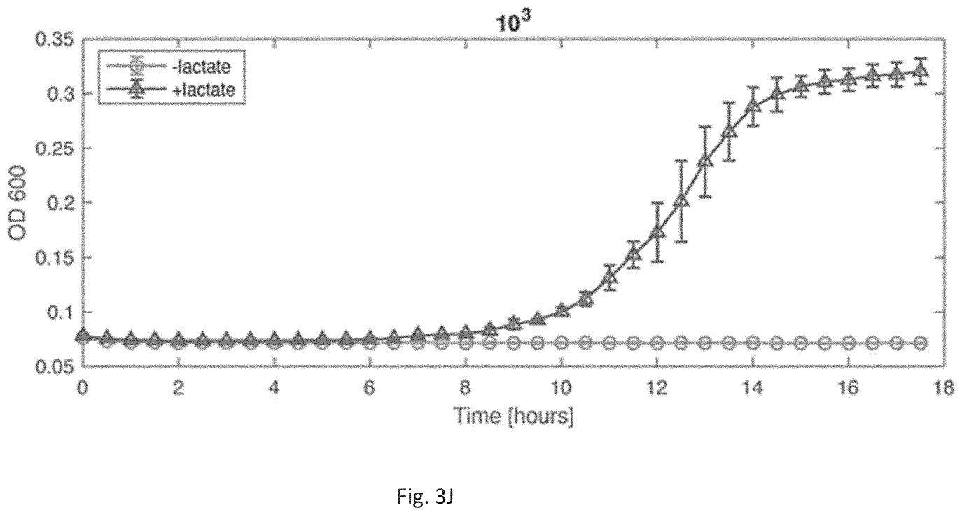

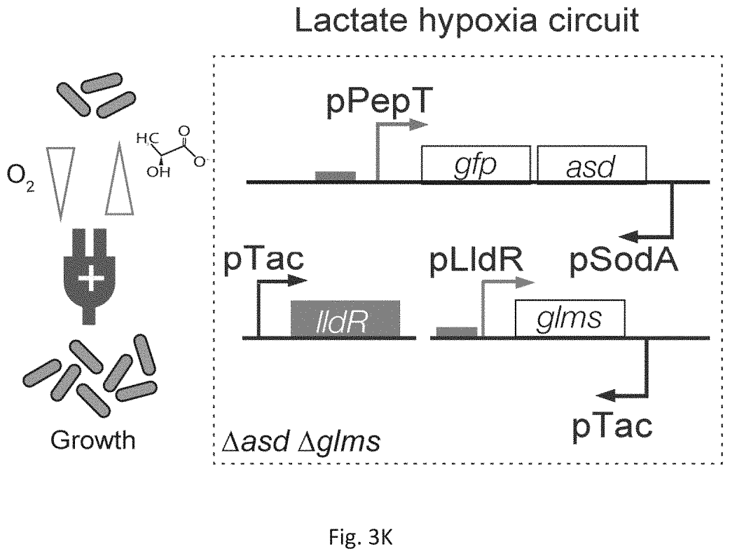

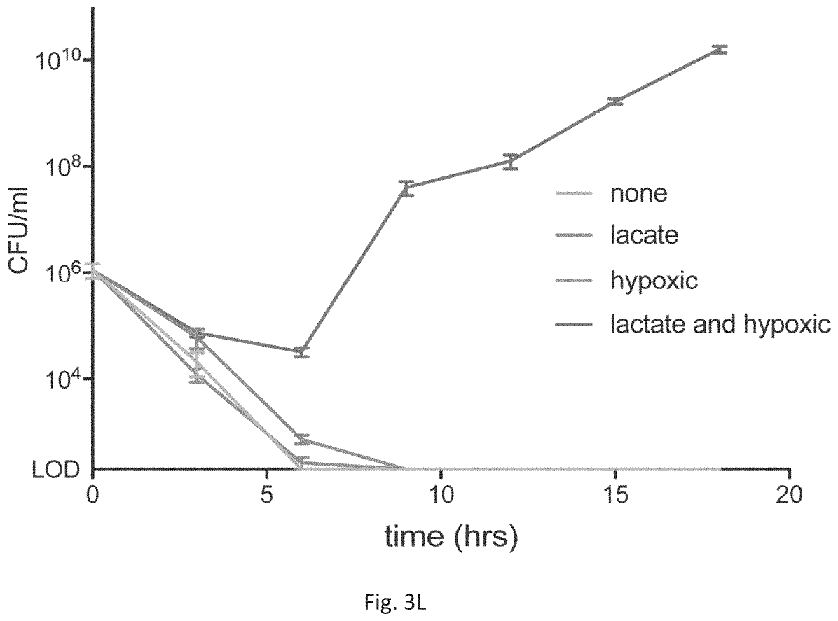

In some embodiments, the hypoxia biosensor circuit includes in addition to the at least one promoter sensitive to a physiological condition and an essential gene of the bacteria under control of the promoter, at least one additional component including but not limited to an additional gene upstream of asd. In some embodiments, the gene is gfp. In some embodiments, the additional gene is genomically integrated. In some embodiments, the hypoxia biosensor circuit also includes an antisense normoxia promoter. In some embodiments, the antisense normoxia promoter pSodA. In some embodiments, the antisense promoter follows the additional gene. In some embodiments, in the hypoxia biosensor circuit is genomically integrated. This optimized circuit design achieved selective bacterial growth in hypoxic conditions is shown in FIG. 3B.

In some embodiments, the lactate biosensor circuit includes in addition to the at least one promoter sensitive to a physiological condition and an essential gene of the bacteria under control of the promoter, at least one additional component. In some embodiments, the ribosome binding strength is lowered or reduced in this circuit. In some embodiments, the lactate biosensor circuit is contained on a plasmid. In some embodiments, the plasmid is a low copy plasmid. This optimized circuit design achieved selective bacterial growth in hypoxic conditions is shown in FIG. 3B.

In some embodiments, the pH biosensor circuit includes in addition to the at least one promoter sensitive to a physiological condition and an essential gene of the bacteria under control of the promoter, at least one additional component including but not limited to a degradation tag. In some embodiments, the degradation tag is downstream from the essential gene. In some embodiment, the degradation tag is LAA. In some embodiments, the ribosome binding strength is lowered or reduced in this circuit. In some embodiments, the lactate biosensor circuit is contained on a plasmid. In some embodiments, the plasmid is a low copy plasmid. This optimized circuit design achieved selective bacterial growth in hypoxic conditions is shown (FIG. 3B).

While optimized engineered biosensor-containing bacteria are exemplified herein, it is within the skill of the art using the guidance set forth herein to more specifically engineer a biosensor-containing bacteria to a particular target by varying the gene expression regulators discussed herein.

A further aspect of the disclosure are biosensor circuits in an AND logic gate. In these embodiments, bacteria are engineered to contain one or more biosensor circuits. Each circuit comprises a different inducible promoter controlling a different essential gene.

In some embodiments, the bacteria will contain more than one biosensor circuit. In some embodiments, the bacteria will contain two biosensor circuits. In some embodiments, the bacteria will contain more than two biosensor circuits. In some embodiments, the bacteria will contain five biosensor circuits.

In some embodiments, the first biosensor circuit and the second biosensor circuit and the third biosensor circuit and the fourth biosensor circuit and the fifth biosensor circuit comprise the same type of inducible promoter, e.g., the promoters are all induced by a physiological condition, controlling a different essential gene.

In some embodiments, the first biosensor circuit and the second biosensor circuit and the third biosensor circuit and the fourth biosensor circuit and the fifth biosensor circuit comprise different types of inducible promoters, e.g., some promoters are induced by a physiological condition, some promoters are induced by bacterial molecules, e.g., autoinducers, and some are induced by exogenous agents, all controlling a different essential gene.

For example, the bacteria could comprise a biosensor comprising a hypoxia sensing promoter and a biosensor comprising a promoter from the LuxR gene, each controlling a different essential gene. In another embodiment, the bacteria could comprise a biosensor comprising a hypoxia sensing promoter and a biosensor comprising a promoter from the LuxR gene and a biosensor comprising a rapamycin-inducible promoter, each controlling a different essential gene. In another embodiment, the bacteria could comprise a biosensor comprising a hypoxia sensing promoter, a biosensor comprising a pH sensing promoter, a biosensor comprising a promoter from the LuxR gene and a biosensor comprising a rapamycin-inducible promoter, each controlling a different essential gene. In yet another embodiment, the bacteria could comprise a biosensor comprising a promoter from the LuxR gene and a biosensor comprising a rapamycin-inducible promoter, each controlling a different essential gene. Thus, it will be appreciated by those of skill in the art that engineered biosensor-containing bacteria can be constructed using a variety of AND combinations which can be tailored to the use of the bacteria.

In some embodiments, the bacteria will comprise a hypoxia biosensor circuit and a lactate biosensor circuit. In some embodiments, the bacteria will comprise a hypoxia biosensor circuit and a pH biosensor circuit. In some embodiments, the bacteria will comprise a lactate biosensor circuit and a pH biosensor circuit. In some embodiments, the bacteria will comprise all three biosensor circuits.

Shown herein was the use of a hypoxia promoter controlling the asd gene and a lactate promoter controlling a gsd gene. The use of both biosensor circuits significantly improved the specificity of the circuits. For example hypoxic environments can be found in the gut and tumor but only lactate is found in a tumor environment. Adding the second lactate biosensor distinguished the bacteria from the two different environments. Bacteria comprising both the hypoxia biosensor circuit and the lactate biosensor circuit were found in tumor tissue only and not in off target organ sites. The pairing of the two biosensor circuits greatly increased tumor targeting specificity.

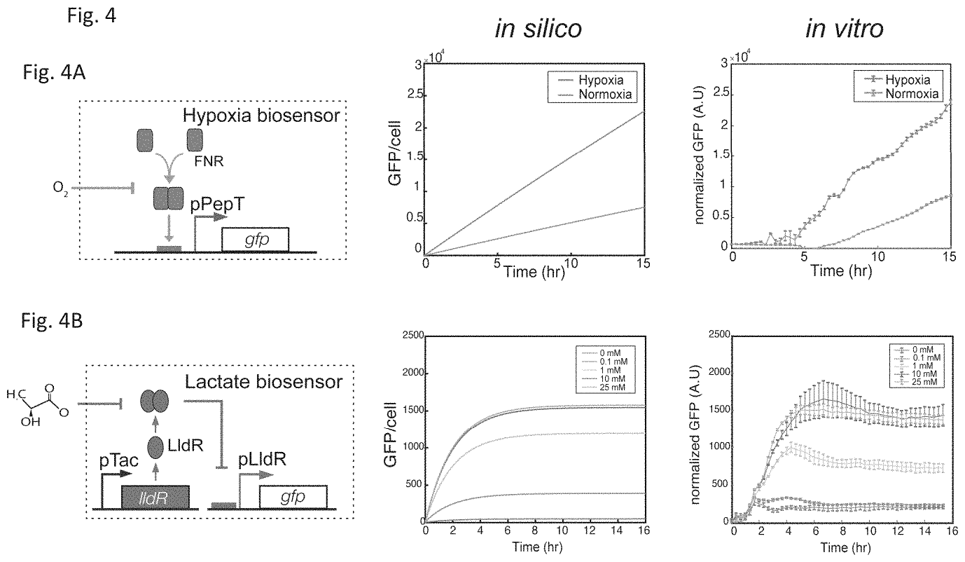

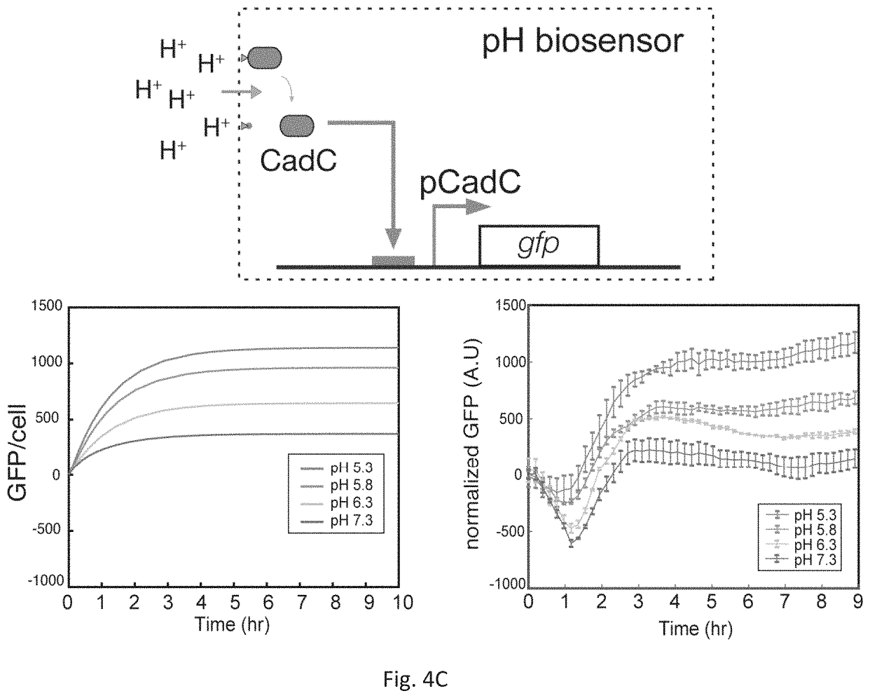

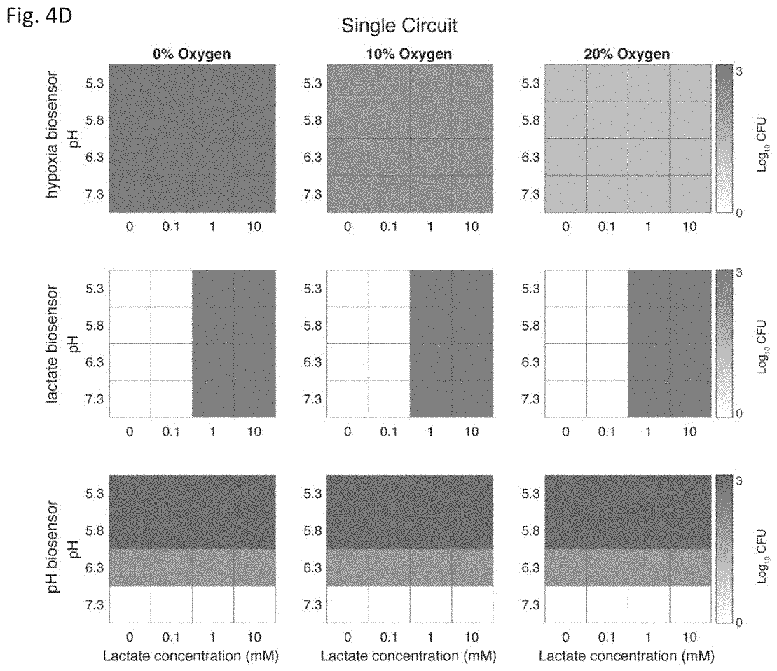

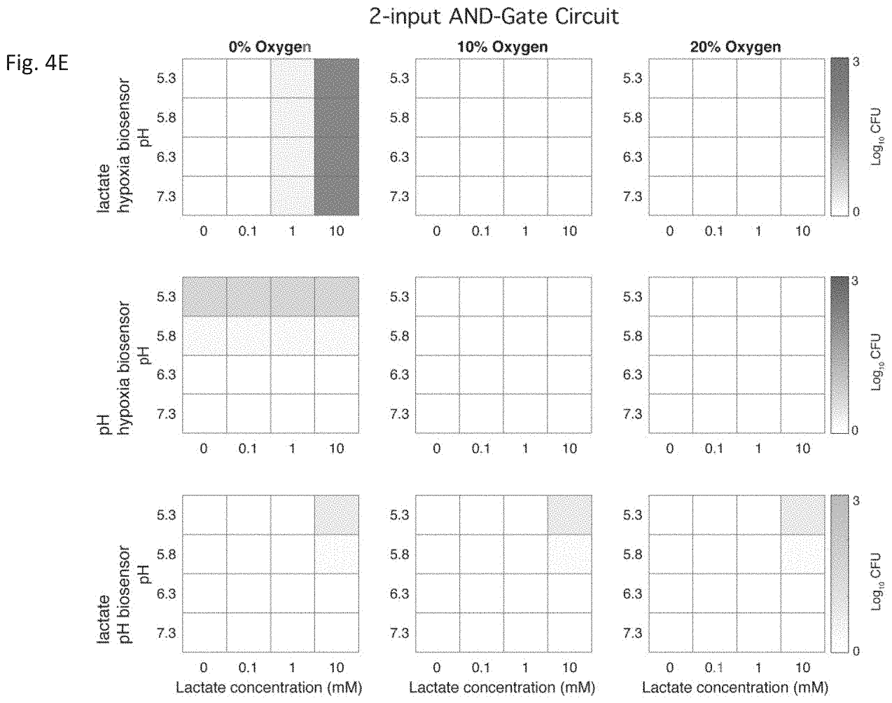

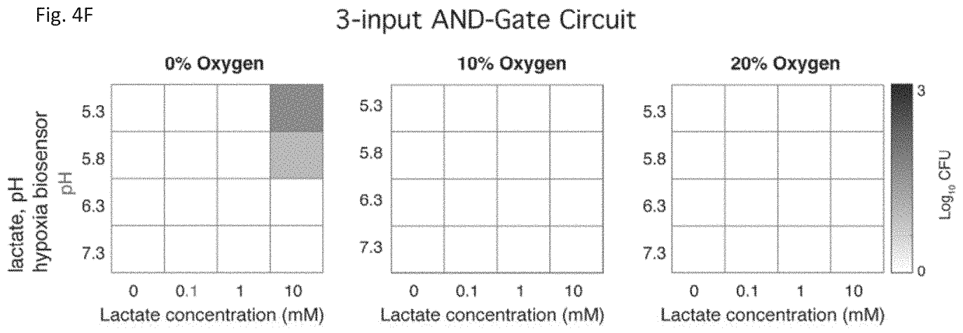

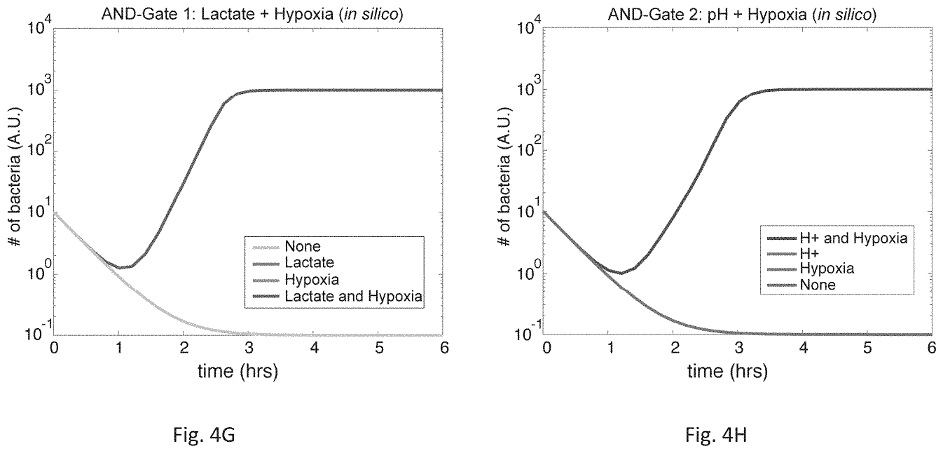

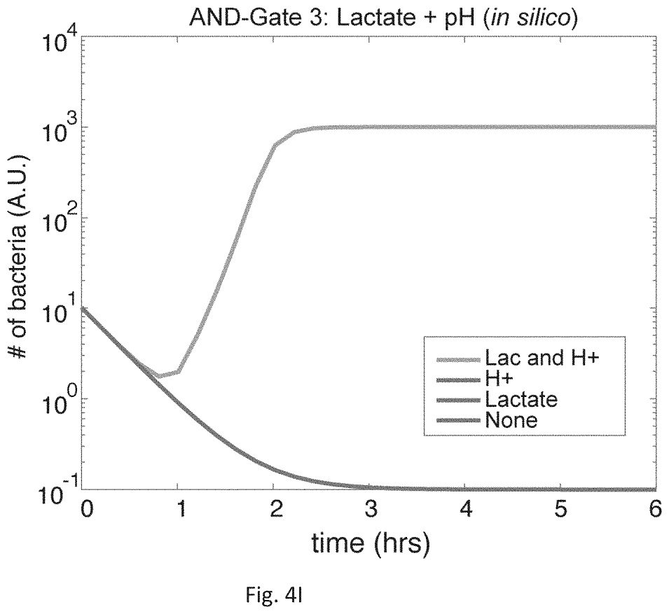

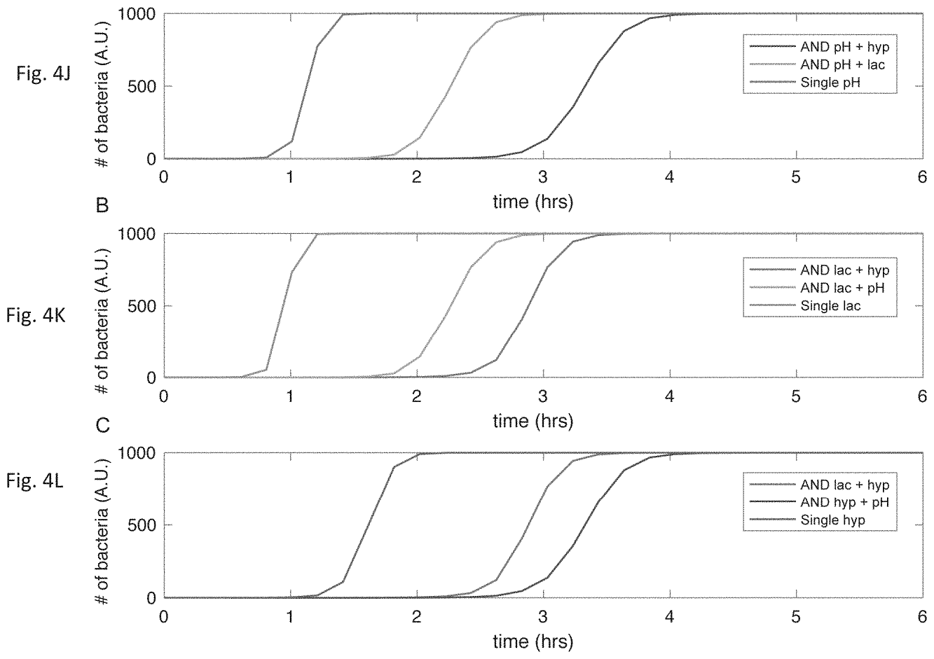

The use of a mathematical model disclosed herein allows the design of multiplexing biosensors across a range of environmental conditions. Briefly, the mathematical model is derived using a system of ordinary differential equations describing the rate of reporter protein production governed by different biosensor regulators, and modulated by lactate, pH, and oxygen conditions The model was fitted to experimental data from in vitro biosensor GFP expression and responses were recapitulated under varied conditions (0-20% oxygen, 0-25 mM lactate and pH 5.3-7.3) (FIGS. 4A-4C). Single and multi-input biosensor circuits (i.e., biosensors driving essential genes) were modeled and experimental conditions for single-input and AND gate circuits. For example, the lactate containment circuit drives maximum bacterial growth at 1-10 mM, the hypoxia containment circuit at 0% oxygen, and pH containment circuit at pH of 5.3-5.8. In the “AND” gate conditions of the 2-input system, growth was captured in selective conditions (high lactate, 0% oxygen), similar to the constructed experimental system. A 3-gate system also demonstrated selective growth in three desired conditions (high lactate, 0% oxygen, and lower pH). See, e.g., Example 4.

Thus, it is within the skill of the art using the methods described herein and the parameters of various organs and tumors as set forth for example in Table 1, to determine which physiological sensing biosensor circuits target which organs or tumors as well as which combinations of physiological sensing biosensor circuits and expression regulators will more efficiently target the desired organs or tumors.

Furthermore, it is within the skill of the art using the methods described herein and the parameters of various organs and tumors, to determine which biosensor circuits comprising other types of inducible promoters target which organs or tumors as well as which combinations of various biosensor circuits and expression regulators will more efficiently target the desired organs or tumors.

Therapeutic and Diagnostic Agents

In some embodiments, the engineered biosensor-containing bacteria described herein further comprise a therapeutic agent. In some embodiments, the therapeutic agent is produced by a plasmid which is introduced into the engineered biosensor-containing bacteria. In some embodiments, the plasmid comprises a nucleic acid encoding a therapeutic agent. In some embodiments, the nucleic acid is operably linked to a promoter. In some embodiments, the promoter is constitutive. In some embodiments, the promoter is inducible.

In some embodiments, the engineered biosensor-containing bacteria described herein further comprise a diagnostic agent. In some embodiments, the diagnostic agent is produced by a plasmid which is introduced into the engineered bio sensor-containing bacteria. In some embodiments, the plasmid comprises a nucleic acid encoding a diagnostic agent. In some embodiments, the nucleic acid is operably linked to a promoter. In some embodiments, the promoter is constitutive. In some embodiments, the promoter is inducible.

Examples of plasmid include but are not limited to pColE1, p15A, pAH162, and pSC101.

Examples of constitutive promoters include but are not limited to a chicken beta actin promoter, a retroviral Rous sarcoma virus (RSV) LTR promoter (optionally with a RSV enhancer), a cytomegalovirus (CMV) promoter (optionally with a CMV enhancer), a SV40 promoter, a dihydrofolate reductase promoter, a 13-actin promoter, a phosphoglycerol kinase (PGK) promoter, and an EF1a promoter (Invitrogen).

Examples of inducible promoters regulated by exogenous agents include but are not limited to a zinc-inducible sheep metallothionine (MT) promoter, a dexamethasone (Dex)-inducible mouse mammary tumor virus (MMTV) promoter, a T7 polymerase promoter system, a ecdysone insect promoter, a tetracycline-repressible system, a tetracycline-inducible system, a RU486-inducible system, and a rapamycin-inducible system.

In some embodiments, the plasmid further comprise other components including but not limited to C-terminal hemagglutinin tag, stabilizing elements (to minimize plasmid loss in vivo), antibiotic resistance genes, and terminators.

Stabilizing elements include but are not limited to the hok/sok system (Gerdes et al. 1986) and alp7 partitioning system (Derman et al. 2012).

Antibiotic resistance genes include but are not limited to resistance to ampicillin, tetracycline and kanamycin

In some embodiments, the plasmid is high copy. In some embodiments, the plasmid is medium copy. In some embodiments, the plasmid is low copy.

In some embodiments, the therapeutic agent is an inhibitor of an immune suppressor molecule, for example, an inhibitor of an immune checkpoint molecule. The immune system is finely regulated to protect from invading pathogens, while avoiding immune responses mounted against the host's own cells. Immune checkpoint molecules help prevent the development of autoimmune diseases. Several cancer drugs aim to inhibit these checkpoints in order to activate the immune system and boost the patient's anti-tumor responses, thus allowing the immune system to mount immune responses against self-antigens on cancerous cells. However, altered immunoregulation can provoke immune dysfunction and lead to autoimmune disorders when administered systemically. The problem of immune dysfunction, e.g., the development of an undesired autoimmune response, can be addressed by delivering an immune checkpoint inhibitor or inhibitor of another immune suppressor molecule locally at the tumor site as accomplished by the programmable bacteria of the invention.

The immune checkpoint molecule to be inhibited can be any known or later discovered immune checkpoint molecule or other immune suppressor molecule. In some embodiments, the immune checkpoint molecule, or other immune suppressor molecule, to be inhibited is selected from TGF-β, CTLA-4, PD-1, PD-L1, PD-L2, TIGIT, VISTA, LAG-3, TIM1, TIM3, CEACAM1, LAIR-1, HVEM, BTLA, CD160, CD200, CD200R, CD39, CD73, CD86, B7-H3, B7-H4, IDO, TDO, KIR, and A2aR.

In some embodiments, the inhibitor of the immune suppressor molecule is an antibody.

In some embodiments, the inhibitor of the immune suppressor molecule is a nanobody. Nanobodies are single single-domain antibodies that are small in size (approximately 15 kDa), maintain strong binding affinity, and can be recombinantly produced in bacteria. These nanobodies address the issues of poor tumor penetration of some large checkpoint inhibitor antibodies in some forms of cancer such as lung. The nanobodies will also minimize immune-related adverse events (IRAE), particularly in combination with locally released LPS from SLC.

In some embodiments, the engineered biosensor-containing bacteria contain plasmids capable of producing one or more agents which inhibit immune checkpoint inhibitors, e.g., one, two, three, four, five, six or more plasmids that produce agents which inhibit immune checkpoint inhibitors.

Thus, a further embodiment is the therapeutic agent is an inhibitor of CD47, including but not limited to antibodies and nanobodies. In further embodiments, the therapeutic agent is an inhibitor of SIRPα, including but not limited to antibodies and nanobodies.

In some embodiments, the therapeutic agent is an immunostimulatory molecule including but not limited to chemokine and cytokines. These molecules include but are not limited to CCL2, CXCL9, CXCL16, IL-18, IL-15, GM-CSF, IL-2, IL-15, IL-12, IL-7, IL-21, TNF, and interferon gamma (IFN-gamma).

In some embodiments, the therapeutic agent is heterodimer comprising subunits from more than one chemokine or cytokine. An example of such a heterodimer is IL12p70, a heterodimer comprised of IL12p40 and IL12p35. In some embodiments, the nucleic acid encoding the heterodimer is on one plasmid.

In yet further embodiments, the therapeutic agent is a toxin. In some embodiments, the toxins derive from bacteria. These toxins would include but are not limited to hemolysin E, melittin, anti-microbial peptides, diphtheria toxins, gelonin toxins, and anthrax toxins.

In a further embodiment, the therapeutic agent is a tumor antigen. As used herein the term “tumor antigen” is meant to refer to tumor-specific antigens, tumor-associated antigens (TAAs), and neoantigens. Tumor antigens are antigenic molecules produced in tumor cells that trigger an immune response in the host. These tumor specific antigens or tumor-associated antigens (TAAs) may be specific to a particular type of cancer cell or tumor cell and therefore the generated immune response will be directed to that cancer or tumor cell type. Examples of tumor antigens are ovalbumin and cytokeratin 19 (CK19). CK19 is a type I cytokeratin that has been suggested to be a suitable antigenic target for eliciting antitumor immunity. Cancer antigenic peptides are listed on databases including the Cancer Research Institute.

Tumor antigens are classified based on their molecular structure and source. Any protein produced in a tumor cell that has an abnormal structure due to mutation can act as a tumor antigen. Mutation of protooncogenes and tumor suppressors which lead to abnormal protein production are the cause of the tumor and thus such abnormal proteins are called tumor-specific antigens. Examples of tumor antigens include products of mutated oncogenes and tumor suppressor genes. Examples of tumor-specific antigens include the abnormal products of ras and p53 genes. Thus, mutated antigens are only expressed by cancer as a result of genetic mutation or alteration in transcription.

In contrast, mutation of other genes unrelated to the tumor formation may lead to synthesis of abnormal proteins which are called tumor-associated antigens. These tumor-associated antigens are the products of other mutated genes that are overexpressed or aberrantly expressed cellular proteins. These overexpressed/accumulated antigens are expressed by both normal and neoplastic tissue, with the level of expression highly elevated in neoplasia. It should be noted that the classifications of “tumor specific antigen” and “tumor associated antigen” or of any of the “classes” described below are not meant to be mutually exclusive, there is overlap between the different “classes” with many tumor antigens falling into more than one “class”; thus the terminology is meant to be a general way of categorizing or grouping tumor antigens based on their characteristics and origin.

Oncofetal antigens are another important class of tumor antigens that are typically only expressed in fetal tissues and in cancerous somatic cells. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens.

In addition to proteins, other substances like cell surface glycolipids and glycoproteins may also have an abnormal structure in tumor cells and could thus be targets of the immune system. Thus, other antigens are altered cell surface glycolipids and glycoproteins that are post translationally altered, e.g., have tumor-associated alterations in glycosylation.

Other examples include tissue differentiation antigens, which are antigens that are specific to a certain type of tissue. Mutant protein antigens are more specific to cancer cells because normal cells do not typically contain these proteins. Normal cells will display the normal protein antigen on their MHC molecules, whereas cancer cells will display the mutant version. Cell type-specific differentiation antigens are lineage-restricted (expressed largely by a single cancer histotype). There are also vascular or stromal specific antigens.

Cancer-testis antigens are expressed only by cancer cells and adult reproductive tissues such as testis and placenta. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Example antigens of this type are CTAG1B and MAGEA1.

Proteins that are normally produced in very low quantities but whose production is dramatically increased in tumor cells, trigger an immune response. An example of such a protein is the enzyme tyrosinase, which is required for melanin production. Normally tyrosinase is produced in minute quantities but its levels are very much elevated in melanoma cells.

In addition to these types of antigens, there are also known neoantigens which can be used to stimulate an immune response. These antitumor T cells recognize unique antigenic features of tumor cells, characterized by mutations that permit uncontrolled growth and/or endow these cells with the ability to metastasize or survive within physiological locations outside of where the tumor initially originated. Such mutated-self, tumor-specific antigens (TSA), termed “neoantigens,” represent the Achilles heel for a developing tumor. In fact, recent data demonstrate immunodominant T cell reactivities directed against mutated neoantigens following PD-1 blockade. Thus, the identification of a patient's unique neoantigen repertoire provides a novel therapeutic avenue through which to promote durable and systemic antitumor immunity.

In some embodiments, the diagnostic agent is a detectable marker or label.

### Methods of Use

The engineered biosensor-containing bacteria described herein have multiple uses. In some embodiments, one type or strain of engineered biosensor-containing bacteria is used. In some embodiments, more than one type or strain of engineered biosensor-containing bacteria is used. In some embodiments, the engineered biosensor-containing bacteria comprises more than one biosensor circuit.

Because the tumor environment is hypoxic, low in pH and high in lactate, one use of the engineered biosensor-containing bacteria is to target bacteria to a tumor, either for treatment or diagnosis. Thus, one embodiment is a method of treating or curing cancer in a subject comprising administering to the subject a therapeutically effective amount of engineered biosensor-containing bacteria or a therapeutically effective amount of a composition or solution comprising engineered biosensor-containing bacteria. In some embodiments, the engineered biosensor-containing bacteria themselves are a therapeutic agent for cancer. In some embodiments, the engineered biosensor-containing bacteria further comprises a therapeutic agent. In some embodiments, engineered biosensor-containing bacteria further comprises a plasmid which produces a therapeutic agent. In some embodiments, the engineered biosensor-containing bacteria comprises more than one biosensor circuit. In some embodiments, the engineered biosensor-containing bacteria comprise different types of inducible promoters, e.g., some promoters are induced by a physiological condition, some promoters are induced by bacterial molecules, e.g., autoinducers, and some are induced by exogenous agents, all controlling a different essential gene. In some embodiments, the engineered biosensor-containing bacteria comprise the same type of inducible promoters, e.g., the promoters are all induced by a physiological condition, controlling a different essential gene. In some embodiments, the engineered biosensor-containing bacteria comprises one or more biosensor circuits that detect hypoxia, low pH and high lactate levels.

Different cancers have different microenvironments, i.e., varying levels of oxygen, pH and lactate. For example, colon and liver cancers have different levels of oxygen, pH and lactate than other cancers. Thus, by fine tuning the biosensor circuits specific cancers can be targeted for treatment. Additionally, engineered biosensor-containing bacteria with an AND logic gate, i.e., more than one biosensor circuit, can more efficiently target the tumor especially when the biosensor circuits are fine tuned for particular physiological signatures of a tumor. As shown herein, multiplexing the biosensor circuits allowed a 100-fold improvement in specificity and containment of the engineered biosensor-containing bacteria. The specificity of the engineered biosensor-containing bacteria will also allow for more specific targeting to the tumor, the biocontainment of the bacteria in the tumor and prevent colonization outside the tumor.

The engineered biosensor-containing bacteria can also be used to diagnose cancer. Thus, one embodiment is a method of diagnosing cancer in a subject comprising administering to the subject an effective amount of engineered biosensor-containing bacteria or an effective amount of a composition or solution comprising engineered biosensor-containing bacteria. In some embodiments, the engineered biosensor-containing bacteria themselves are a diagnostic agent for cancer. In some embodiments, the engineered biosensor-containing bacteria also comprises a diagnostic agent. In some embodiments, engineered biosensor-containing bacteria further comprises a plasmid which produces a diagnostic agent. In some embodiments, the engineered biosensor-containing bacteria comprises more than one biosensor circuit. In some embodiments, the engineered biosensor-containing bacteria comprises one or more biosensor circuits that detect hypoxia, low pH and high lactate levels. It is contemplated that the use of the engineered biosensor-containing bacteria for diagnosis of cancer would be at an earlier time point than traditional imaging diagnosis.

As stated above, different cancers have different microenvironments, including different oxygen levels, pH levels and lactate levels. Again by fine tuning the sensitivity and specificity of the engineered biosensor-containing bacteria, using multiplexing, ribosome binding sites, gene copy, etc., the engineered biosensor-containing bacteria can not only diagnose a cancer but also diagnose the location and type of cancer giving more information for treatment depending on where in the body and tumor the engineered biosensor-containing bacteria with varying sensitivity and specificity colonizes.

Certain genetic mutations in cancers have particular microenvironment signatures. Using the engineered biosensor-containing bacteria can correlate a patient's cancer to a genetic mutation without invasive and lengthy genetic testing. By fine tuning the engineered biosensor-containing bacteria to colonize in the particular oxygen, pH and/or lactate conditions correlated to the genetic mutation it can be determined whether the cancer corresponds to this mutation.

One method of using the engineered biosensor-containing bacteria to diagnose cancer would be to also engineer the bacteria to have a means of detection which is not harmful to a patient.

A further method of using the engineered biosensor-containing bacteria to diagnose cancer would be to also engineer the engineered biosensor-containing bacteria to produce an enzyme when it reached the tumor, which would be excreted in a patient's urine in about a day.

The terms “cancer”, “tumor”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include melanoma, lung cancer, head and neck cancer, renal cell cancer, colon cancer, colorectal cancer, squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, endometrial carcinoma, myeloma (such as multiple myeloma), salivary gland carcinoma, kidney cancer such as renal cell carcinoma and Wilms' tumors, basal cell carcinoma, prostate cancer, vulval cancer, thyroid cancer, testicular cancer, and esophageal cancer.

A “tumor” refers to the mass of tissue formed as cancerous cells grow and multiply, which can invade and destroy normal adjacent tissues. Cancer cells can break away from a malignant tumor and enter the bloodstream or lymphatic system, such that cancer cells spread from the primary tumor to form new tumors in other organs.

A “solid tumor” refers to an abnormal growth or mass of tissue that usually does not contain cysts or liquid areas. Solid tumors may be benign (not cancerous) or malignant (cancerous). Different types of solid tumors are named for the type of cells that form them. Examples of solid tumors are sarcomas, carcinomas, and lymphomas. Leukemias (cancers of the blood) generally do not form solid tumors

Non-limiting examples of preferred cancers for treatment or cure by the engineered biosensor-containing bacteria include breast, melanoma (e.g., metastatic malignant melanoma), renal cancer (e.g., clear cell carcinoma), prostate cancer (e.g., hormone refractory prostate adenocarcinoma), pancreatic adenocarcinoma, colon cancer (CRC), lung cancer (e.g., non-small cell lung cancer), esophageal cancer, squamous cell carcinoma of the head and neck, liver cancer, ovarian cancer, cervical cancer, thyroid cancer, glioblastoma, glioma, and more generally solid tumors. Additionally, the engineered biosensor-containing bacteria can be used to treat refractory or recurrent malignancies whose growth may be inhibited using compositions described herein.

Organs in the body when healthy and when diseased have different physiological signatures. In particular, the gut including the various portions the small intestine, e.g, upper small intestine track (S1) and lower small intestine track (S2), the large intestine, e.g., upper large intestine track (L1) and lower large intestine track (L2), and the caecum, have different physiological conditions. There is a gradient of oxygen levels and pH levels in these areas of the gut. By leveraging this knowledge, the engineered biosensor-containing bacteria can be used for targeting areas of the gut for both treatment and diagnosis. For example there was a 10-fold enrichment of hypoxia dependent engineered bio sensor-containing bacteria in the large intestine as compared to the small intestine. There is an increase in colonization of pH sensing engineered biosensor-containing bacteria in the upper GI tract where the pH is higher. The engineered biosensor-containing bacteria can further be fine-tuned to more specifically target a particular area of the gastrointestinal tract. In addition to providing precise targeting, it also ensures safety. For example, in cases where patients have weakened immune system or bleached epithelial barrier, engineered biosensor-containing bacteria are contained in desired region. In these some embodiments of these methods, engineered biosensor-containing bacteria comprising more than one biosensor circuit can be used. In some embodiments of these methods, more than one type of engineered biosensor-containing bacteria can be used. For example, both a first engineered biosensor-containing bacteria comprising a hypoxic sensing biosensor circuit can be used in conjunction with a second engineered biosensor-containing bacteria comprising a pH sensing biosensor circuit.

Thus, a further embodiment is a method of treating a disease or condition in the large intestine in a subject comprising administering to the subject a therapeutically effective amount of engineered biosensor-containing bacteria which detects hypoxia or a therapeutically effective amount of a composition or solution comprising engineered biosensor-containing bacteria which detects hypoxia. In some embodiments, the engineered biosensor-containing bacteria themselves are a therapeutic agent. In some embodiments, the engineered biosensor-containing bacteria also comprises a therapeutic agent. In some embodiments, engineered biosensor-containing bacteria further comprises a plasmid which produces a therapeutic agent.

A further embodiment is a method of diagnosing a disease or condition in the large intestine in a subject comprising administering to the subject an effective amount of engineered biosensor-containing bacteria which detects hypoxia or an effective amount of a composition or solution comprising engineered biosensor-containing bacteria which detects hypoxia. In some embodiments, the engineered biosensor-containing bacteria themselves are a diagnostic agent. In some embodiments, the engineered biosensor-containing bacteria also comprises a diagnostic agent. In some embodiments, engineered biosensor-containing bacteria further comprises a plasmid which produces a diagnostic agent. One method of using the engineered biosensor-containing bacteria to diagnose disease or conditions would be to also engineer the bacteria to have a means of detection which is not harmful to a patient.

Yet a further embodiment is a method of treating a disease or condition in the small intestine in a subject comprising administering to the subject a therapeutically effective amount of engineered biosensor-containing bacteria which detects high pH or a therapeutically effective amount of a composition or solution comprising engineered biosensor-containing bacteria which detects high pH. In some embodiments, the engineered biosensor-containing bacteria themselves are a therapeutic agent. In some embodiments, the engineered biosensor-containing bacteria also comprises a therapeutic agent. In some embodiments, engineered biosensor-containing bacteria further comprises a plasmid which produces a therapeutic agent.

A further embodiment is a method of diagnosing a disease or condition in the small intestine in a subject comprising administering to the subject an effective amount of engineered biosensor-containing bacteria which detects high pH or an effective amount of a composition or solution comprising engineered biosensor-containing bacteria which detects high pH. In some embodiments, the engineered biosensor-containing bacteria themselves are a diagnostic agent. In some embodiments, the engineered biosensor-containing bacteria also comprises a diagnostic agent. In some embodiments, engineered biosensor-containing bacteria further comprises a plasmid which produces a diagnostic agent. One method of using the engineered biosensor-containing bacteria to diagnose disease or conditions would be to also engineer the bacteria to have a means of detection which is not harmful to a patient.

In some embodiments, these methods can be combined and the use of engineered biosensor-containing bacteria comprising a hypoxia-sensing promoter can be used in combination with engineered biosensor-containing bacteria comprising a pH sensing promoter.

This approach can also be used in the skin. Skin has different levels of physiological signatures when healthy and when wounded. For example, the engineered biosensor-containing bacteria can be used for targeted delivery of healing agents to wounded skin, which in some cases may not outwardly appear to be wounded. The engineered biosensor-containing bacteria could also be used for treating and diagnosing skin cancers, the latter at earlier stages than conventional skin cancer diagnosis. This could be accomplished administering to the skin a solution or composition of engineered biosensor-containing bacteria which target skin cancer physiological signatures and looking for colonization at particular areas of the skin.

In addition to diseases, because biosensor can target specific location within the body it can be used to sense and monitor environmental changes at the region overtime.

### Administration, Dosing and Pharmaceutical Compositions

As discussed above, the present disclosure provides a pharmaceutical composition comprising a therapeutically effective amount of engineered biosensor-containing bacteria in any form described herein and a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

The pharmaceutical compositions may be formulated in a conventional manner using one or more physiologically acceptable carriers comprising excipients and auxiliaries, which facilitate processing of the active ingredients into compositions for pharmaceutical use. Methods of formulating pharmaceutical compositions are known in the art (see, e.g., “Remington's Pharmaceutical Sciences,” Mack Publishing Co., Easton, Pa.). In some embodiments, the pharmaceutical compositions are subjected to tabletting, lyophilizing, direct compression, conventional mixing, dissolving, granulating, levigating, emulsifying, encapsulating, entrapping, or spray drying to form tablets, granulates, nanoparticles, nanocapsules, microcapsules, microtablets, pellets, or powders, which may be enterically coated or uncoated. Appropriate formulation depends on the route of administration.

The engineered biosensor-containing bacteria may be formulated into pharmaceutical compositions in any suitable dosage form (e.g., liquids, capsules, sachet, hard capsules, soft capsules, tablets, enteric coated tablets, suspension powders, granules, or matrix sustained release formations for oral administration) and for any suitable type of administration (e.g., oral, topical, injectable, intravenous, sub-cutaneous, intratumoral, peritumor, immediate-release, pulsatile-release, delayed-release, or sustained release).

Suitable dosage amounts for the engineered biosensor-containing bacteria may range from about 10⁴ to 10¹² bacteria. The composition may be administered once or more daily, weekly, or monthly. The composition may be administered before, during, or following a meal. In one embodiment, the pharmaceutical composition is administered before the subject eats a meal. In one embodiment, the pharmaceutical composition is administered currently with a meal. In one embodiment, the pharmaceutical composition is administered after the subject eats a meal.

The engineered biosensor-containing bacteria may be administered intravenously, e.g., by infusion or injection. Alternatively, the engineered biosensor-containing bacteria may be administered intratumorally and/or peritumorally. In other embodiments, the engineered biosensor-containing bacteria may be administered intra-arterially, intramuscularly, or intraperitoneally. In some embodiments, the engineered biosensor-containing bacteria colonize about 20%, 30%, 40%, 50%, 60%, 70%, 80%, 90% or more of the tumor. In some embodiments, the engineered biosensor-containing bacteria are co-administered with a PEGylated form of rHuPH20 (PEGPH20) or other agent in order to destroy the tumor septae in order to enhance penetration of the tumor capsule, collagen, and/or stroma.

The engineered biosensor-containing bacteria may be administered via intratumoral injection, resulting in bacteria that is directly deposited within the target tumor.

Depending on the location, tumor type, and tumor size, different administration techniques may be used, including but not limited to, cutaneous, subcutaneous, and percutaneous injection, therapeutic endoscopic ultrasonography, or endobronchial intratumor delivery. In some embodiment, other techniques, such as laproscopic or open surgical techniques are used to access the target tumor.

The dose to be injected is derived from the type and size of the tumor. The dose of the engineered biosensor-containing bacteria is typically lower, e.g., orders of magnitude lower, than a dose for systemic intravenous administration.

The volume injected into each lesion is based on the size of the tumor. To obtain the tumor volume, a measurement of the largest plane can be conducted. The estimated tumor volume can then inform the determination of the injection volume as a percentage of the total volume. For example, an injection volume of approximately 20-40% of the total tumor volume can be used.

In some embodiments, the treatment regimen will include one or more intratumoral administrations. In some embodiments, a treatment regimen will include an initial dose, which followed by at least one subsequent dose. One or more doses can be administered sequentially in two or more cycles.

For example, a first dose may be administered at day 1, and a second dose may be administered after 1, 2, 3, 4, 5, 6, days or 1, 2, 3, or 4 weeks or after a longer interval.

Additional doses may be administered after 1, 2, 3, 4, 5, 6, days or after 1, 2, 3, or 4 weeks or longer intervals. In some embodiments, the first and subsequent administrations have the same dosage. In other embodiments, different doses are administered. In some embodiments, more than one dose is administered per day, for example, two, three or more doses can be administered per day.

The engineered biosensor-containing bacteria may be administered orally and formulated as tablets, pills, dragees, capsules, liquids, gels, syrups, slurries, and suspensions. Pharmacological compositions for oral use can be made using a solid excipient, optionally grinding the resulting mixture, and processing the mixture of granules, after adding suitable auxiliaries if desired, to obtain tablets or dragee cores. Suitable excipients include, but are not limited to, fillers such as sugars, including lactose, sucrose, mannitol, or sorbitol; cellulose compositions such as maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP) or polyethylene glycol (PEG). Disintegrating agents may also be added, such as cross-linked polyvinylpyrrolidone, agar, alginic acid or a salt thereof such as sodium alginate.

Tablets or capsules can be prepared by conventional means with pharmaceutically acceptable excipients such as binding agents (e.g., pregelatinised maize starch, polyvinylpyrrolidone, hydroxypropyl methylcellulose, carboxymethylcellulose, polyethylene glycol, sucrose, glucose, sorbitol, starch, gum, kaolin, and tragacanth); fillers (e.g., lactose, microcrystalline cellulose, or calcium hydrogen phosphate); lubricants (e.g., calcium, aluminum, zinc, stearic acid, polyethylene glycol, sodium lauryl sulfate, starch, sodium benzoate, L-leucine, magnesium stearate, talc, or silica); disintegrants (e.g., starch, potato starch, sodium starch glycolate, sugars, cellulose derivatives, silica powders); or wetting agents (e.g., sodium lauryl sulphate). The tablets may be coated by methods well known in the art. A coating shell may be present, and common membranes include, but are not limited to, polylactide, polyglycolic acid, polyanhydride, other biodegradable polymers, alginate-polylysine-alginate (APA), alginate-polymethylene-co-guanidine-alginate (A-PMCG-A), hydroymethylacrylate-methyl methacrylate (HEMA-MMA), multilayered HEMA-MMA-MAA, polyacrylonitrilevinylchloride (PAN-PVC), acrylonitrile/sodium methallylsulfonate (AN-69), polyethylene glycol/poly pentamethylcyclopentasiloxane/polydimethylsiloxane (PEG/PD5/PDMS), poly N,N-dimethyl acrylamide (PDMAAm), siliceous encapsulates, cellulose sulphate/sodium alginate/polymethylene-co-guanidine (CS/A/PMCG), cellulose acetate phthalate, calcium alginate, k-carrageenan-locust bean gum gel beads, gellan-xanthan beads, poly(lactide-co-glycolides), carrageenan, starch poly-anhydrides, starch polymethacrylates, polyamino acids, and enteric coating polymers.

In some embodiments, the engineered biosensor-containing bacteria are enterically coated for release into the gut or a particular region of the gut, for example, the large intestine. The typical pH profile from the stomach to the colon is about 1-4 (stomach), 5.5-6 (duodenum), 7.3-8.0 (ileum), and 5.5-6.5 (colon). In some diseases, the pH profile may be modified. In some embodiments, the coating is degraded in specific pH environments in order to specify the site of release. In some embodiments, at least two coatings are used. In some embodiments, the outside coating and the inside coating are degraded at different pH levels.

In some embodiments, enteric coating materials may be used, in one or more coating layers (e.g., outer, inner and/o intermediate coating layers). Enteric coated polymers remain unionised at low pH, and therefore remain insoluble. But as the pH increases in the gastrointestinal tract, the acidic functional groups are capable of ionisation, and the polymer swells or becomes soluble in the intestinal fluid.

In another embodiment, the pharmaceutical composition comprising the engineered biosensor-containing bacteria may be a comestible product, for example, a food product. In one embodiment, the food product is milk, concentrated milk, fermented milk (yogurt, sour milk, frozen yogurt, lactic acid bacteria-fermented beverages), milk powder, ice cream, cream cheeses, dry cheeses, soybean milk, fermented soybean milk, vegetable-fruit juices, fruit juices, sports drinks, confectionery, candies, infant foods (such as infant cakes), nutritional food products, animal feeds, or dietary supplements. In one embodiment, the food product is a fermented food, such as a fermented dairy product. In one embodiment, the fermented dairy product is yogurt. In another embodiment, the fermented dairy product is cheese, milk, cream, ice cream, milk shake, or kefir. In another embodiment, the engineered biosensor-containing bacteria are combined in a preparation containing other live bacterial cells intended to serve as probiotics. In another embodiment, the food product is a beverage. In one embodiment, the beverage is a fruit juice-based beverage or a beverage containing plant or herbal extracts. In another embodiment, the food product is a jelly or a pudding. Other food products suitable for administration of the engineered biosensor-containing bacteria of the invention are well known in the art.

In some embodiments, the composition is formulated for intraintestinal administration, intrajejunal administration, intraduodenal administration, intraileal administration, gastric shunt administration, or intracolic administration, via nanoparticles, nanocapsules, microcapsules, or microtablets, which are enterically coated or uncoated. The pharmaceutical compositions may also be formulated in rectal compositions such as suppositories or retention enemas, using, e.g., conventional suppository bases such as cocoa butter or other glycerides. The compositions may be suspensions, solutions, or emulsions in oily or aqueous vehicles, and may contain suspending, stabilizing and/or dispersing agents.

The engineered biosensor-containing bacteria may be administered intranasally, formulated in an aerosol form, spray, mist, or in the form of drops, and conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). Pressurized aerosol dosage units may be determined by providing a valve to deliver a metered amount. Capsules and cartridges (e.g., of gelatin) for use in an inhaler or insufflator may be formulated containing a powder mix of the compound and a suitable powder base such as lactose or starch.

The engineered biosensor-containing bacteria may be administered and formulated as depot preparations. Such long acting formulations may be administered by implantation or by injection, including intravenous injection, subcutaneous injection, local injection, direct injection, or infusion. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

In some embodiments, disclosed herein are pharmaceutically acceptable compositions in single dosage forms. Single dosage forms may be in a liquid or a solid form. Single dosage forms may be administered directly to a patient without modification or may be diluted or reconstituted prior to administration. In certain embodiments, a single dosage form may be administered in bolus form, e.g., single injection, single oral dose, including an oral dose that comprises multiple tablets, capsule, pills, etc. In alternate embodiments, a single dosage form may be administered over a period of time, e.g., by infusion.

Single dosage forms of the pharmaceutical composition may be prepared by portioning the pharmaceutical composition into smaller aliquots, single dose containers, single dose liquid forms, or single dose solid forms, such as tablets, granulates, nanoparticles, nanocapsules, microcapsules, microtablets, pellets, or powders, which may be enterically coated or uncoated. A single dose in a solid form may be reconstituted by adding liquid, typically sterile water or saline solution, prior to administration to a patient.

In other embodiments, the composition can be delivered in a controlled release or sustained release system. In one embodiment, a pump may be used to achieve controlled or sustained release. In another embodiment, polymeric materials can be used to achieve controlled or sustained release of the therapies of the present disclosure (see e.g., U.S. Pat. No. 5,989,463). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. The polymer used in a sustained release formulation may be inert, free of leachable impurities, stable on storage, sterile, and biodegradable. In some embodiments, a controlled or sustained release system can be placed in proximity of the prophylactic or therapeutic target, thus requiring only a fraction of the systemic dose. Any suitable technique known to one of skill in the art may be used.

The routes of administration and dosages described are intended only as a guide. The optimum route of administration and dosage can be readily determined by a skilled practitioner. The dosage may be determined according to various parameters, especially according to the location of the tumor, the size of the tumor, the age, weight and condition of the patient to be treated and the route and method of administration.

### Kits

The present invention also provides kits comprising any of the engineered biosensor-containing bacteria described herein.

A kit includes one or more components including, but not limited to, engineered biosensor-containing bacteria described herein. Kits may further include a pharmaceutically acceptable carrier, as discussed herein. The engineered biosensor-containing bacteria can be formulated as a pure composition or in combination with a pharmaceutically acceptable carrier, in a pharmaceutical composition.

In some embodiments, a kit includes engineered biosensor-containing bacteria described herein in one container (e.g., in a sterile glass or plastic vial).

In some embodiments, a kit includes engineered biosensor-containing bacteria described herein in one container (e.g., in a sterile glass or plastic vial) and a second or subsequent engineered biosensor-containing bacteria described herein in another container (e.g., in a sterile glass or plastic vial).

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding the engineered biosensor-containing bacteria may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

### EXAMPLES

This invention will be better understood from the Experimental Details, which follow. However, one skilled in the art will readily appreciate that the specific methods and results discussed are merely illustrative of the invention as described more fully in the claims that follow thereafter.

### Example 1—Materials and Methods

Host strains and culturing. ELH1301 was kindly provided by Dr. Elizabeth Hohmann. E. coli Nissle 1917 (EcN) was obtained from the Bhatia lab (Riglar and Silver 2018). For full strain information, please refer to Table 3. All bacteria were cultured in LB media (Sigma-Aldrich) with appropriate antibiotic selection (100 μg ml⁻¹ ampicillin, 50 μg ml⁻¹ kanamycin, 25 μg ml⁻¹ chloramphenicol) at 37° C.

Plasmids and biosensor library constructions. Plasmids were constructed using Gibson Assembly or using standard restriction digest and ligation cloning and transformed into Mach1 competent cells (Invitrogen). The biosensors were constructed by synthesizing promoters from IDT, except for the pPepT, pLldR and pCadC promoters obtained via colony PCR from EcN. Promoters were cloned in front of the sfGFP gene of a previously used ColE1 pTD103 sfGFP plasmid (Ruder et al. 2011). To construct biocontainment circuits, essential genes asd or glms were added after the sfGFP gene. To tune the circuit sensitivity, gene copy numbers (colE1, p15a, or sc101 replication origins and single genome integration), antisense promoters, ribosome binding sites, and protein-degradation tags were engineered by cloning each segment using synthesized DNA followed by Gibson Assembly. A detailed table of biosensor plasmids is provided (Table 2).

Chromosomal gene deletion and integration in bacteria. Essential genes asd and glms were deleted using the λ-Red recombination system (Khalil and Collins 2010). Linear DNA with ploxp-cmR-loxp template were PCR amplified using pkD3 plasmid and electroporated into bacteria carrying pKD46 plasmid. Bacteria were recovered and plated with supplement DAP and D-glucosamine. Chromosomal deletions of the essential genes were verified by PCR and sequencing. To integrate biosensor circuits into the bacterial genome, the CRIM plasmid system was employed (Kitada et al. 2018). Plasmid pAH162 carrying tetracycline resistance gene was used to integrate the construct at φ80 sites. (Khalil and Collins 2010). Hypoxia promoter driving asd gene was cloned into the plasmid followed by genomic integration. Integration was verified using PCR and sequencing.

Characterization of biosensors in vitro. Each bacterial strain was grown in liquid culture overnight, then used to inoculate induction experiments. For hypoxia biosensors, each variant was cultured overnight in normoxic and hypoxic conditions. Hypoxia was achieved by growing bacteria in BD GasPak™ EZ anaerobic pouches and static culture in 37° C. For lactate biosensors, each lactate biosensor variant strain was grown in 6 wells of a 24-well Qiagen Block overnight in LB broth with relevant antibiotic and at lactic acid concentrations of 0, 0.1, 1, 5, and 10 mM. For pH biosensors, each variant was cultured in 96-well plates with pH roughly ranging from 4.4-8. Negative controls of untransformed Mach1 and Nissle cells, along with a positive control of pTac sfGFP, a strain that expresses sfGFP using the synthetic Tac promoter were grown in the same conditions. All cultures were started at a bacterial OD₆₀₀ of 0.1. After 16-20 hours of growth, fluorescence and absorbance data were collected using a Tecan Infinite MicroPlate reader.

Characterization of biocontainment circuit in vitro. All containment strains were grown in LB media overnight with supplements (DAP and/or D-glucosamine) added. Cultures were then washed three times with PBS to remove residual supplements, followed by serial 10-fold dilutions ten times into LB with inducers (10 mM lactate, pH 5.5, 6, 7 or cultured in hypoxia). All variants were cultured for 12-16 hours and plated on LB agar plates with supplements and colony forming units were calculated the next day.

Biosensor in vitro characterization data analysis. All biosensor in vitro fluorescence signals were calculated by dividing raw GFP pixel intensity by OD₆₀₀ value, both obtained from plate reader data. The background fluorescence signal (no plasmid control of the same strain) was subtracted. For the hypoxia biosensor, fluorescence signal was normalized to constitutive promoter (pTac) control, to account for protein folding maturation under hypoxia, as has been observed (Riglar et al. 2017). The fold change of each biosensor was quantified as the ratio between normalized fluorescence signal of induced and non-induced conditions. All triplicate values were averaged. The baseline condition was at 20% oxygen concentration, 0 mM lactate concentration, and 7.3 pH.

Mammalian cell culture. 393T5, 373T1, 802T1, 482T1, 368T1, and 393T1 cell lines were kindly provided by Dr. Tyler Jacks. All other cell lines were obtained from ATCC. Mammalian cells were cultured in DMEM/F-12 media with GlutaMAX supplement (Gibco; for 393T5, 373T1, 802T1, 482T1, 368T1, and 393T1) or RPMI 1640 media (Gibco; for CT26, 4T1, A20 and 368T1) and supplemented with 10% fetal bovine serum (FBS, Gibco) and 1% penicillin/streptomycin (CellGro), Human Lung Fibroblast (HLF) were cultured in Fibroblast Growth Kit-Low serum (ATCC PCS-201-041) with 2% FBS and placed inside a tissue culture incubator at 37° C. maintained at 5% CO₂. For full cell line information, refer to Table 2.

Bacteria culture with monolayer cell supernatants. All cell lines were seeded in 9 identical wells on 6-well plates in 3 mL of DMEM/10% FBS with Sodium Bicarbonate or RPMI/10% FBS medium without buffer at an initial cell count of 100 k. Twice daily over the following 5 days, in 8-12-hour intervals, the entire contents of each well were removed and transferred to a 15 mL tube. The sample was centrifuged at 200 rcf for 5 minutes, and the media supernatant was collected into another tube, which was frozen in −80° C. to preserve lactate concentration at the time of collection. After collection, each monolayer cell supernatant was separately incubated with the 3 biosensors, negative control and positive control. Hypoxia biosensor was tested with varying level of oxygen in a culturing chamber connected to the CO₂—O₂ controller (Okolab). All bacteria were grown for 12-16 hours and assayed for fluorescence and absorbance with the Tecan Infinite MicroPlate reader.

Bacteria coculture with tumor spheroids. Tumor spheroids were generated by seeding cells in round-bottom ultra-low attachment 96-well plate (Corning). Each well contains 2,500 CT26 cells in 100 μl of RPMI/10% FBS medium without antibiotics. The plate was centrifuged at 3,000 rcf for 5 minutes to aggregate cells at the bottom of the plate and placed inside a tissue culture incubator for 4 days prior to coculture with bacteria.

Bacteria were cultured in a 37° C. shaker overnight to reach stationary phase before use. 10⁶ CFU S. typhimurium were inoculated into wells containing 4-day mature tumor spheroids and placed back into the tissue culture incubator. After 2 hours of bacteria inoculation, media was removed. Tumor spheroids were washed with 200 μl of PBS repeatedly while leaving spheroids at the bottom of plate. After washing, 200 μl of media containing 2.5 μg/ml gentamicin (Gibco) was added, and tumor spheroids were monitored for growth. Acquisition of spheroid still images was performed with EVOS FL Auto 2 Cell Imaging Systems. The scope and accessories were programmed using the Celleste Imaging Analysis software.

Bacterial colonization quantification via colony counts. Spheroids containing bacteria were repeatedly washed with 200 μl of PBS, while leaving spheroids at the bottom of plate. After washing, spheroids were re-suspended in 100 μl of PBS and homogenized using mechanical dissociation with sterile tips and repeated pipetting. Destruction of spheroids were confirmed by microscopy. Serial 10-fold dilutions of the samples were inoculated on appropriate agar plates.

GFP average fluorescence and radial histograms for spheroids. To measure the spatiotemporal dynamics of bacteria invading tumor spheroids, a threshold brightness value for each TL image was first found to distinguish the dark spheroid from the light background. Scikit-image implementations of two popular thresholding methods were used: the minimum method (Daeffler et al. 2017) for images taken daily, and Yen's method (Hwang et al. 2017) for other images. The largest region within the resulting threshold-based image mask was identified as the tumor spheroid, and the mean intensity of sfGFP fluorescence within this region was calculated. To compute radial histograms, mean sfGFP fluorescence for many thin annuli with variable mean radii were measured and centered on the centroid of the spheroid mask region.

Animal gastrointestinal models. All animal experiments were approved by the Institutional Animal Care and Use Committee (Columbia University, protocol AC-AAAN8002). Animal experiments were performed on 4-6 week old BALB/c mice (Taconic Biosciences) after pretreatment with antibiotics (Ampicillin and Neomycin) for 7-10 days prior to oral gavage of bacterial strains (Mao et al. 2018). Fecal matter was collected daily and plated on selective plates with or without DAP to quantify containment. Animals were euthanized at an endpoint of 7 days, and the whole gut was excised, separated into 5 sections (S1, S2, C, L1 and L2), weighted and then homogenized to plate on DAP supplemented plates for colony count the next day.

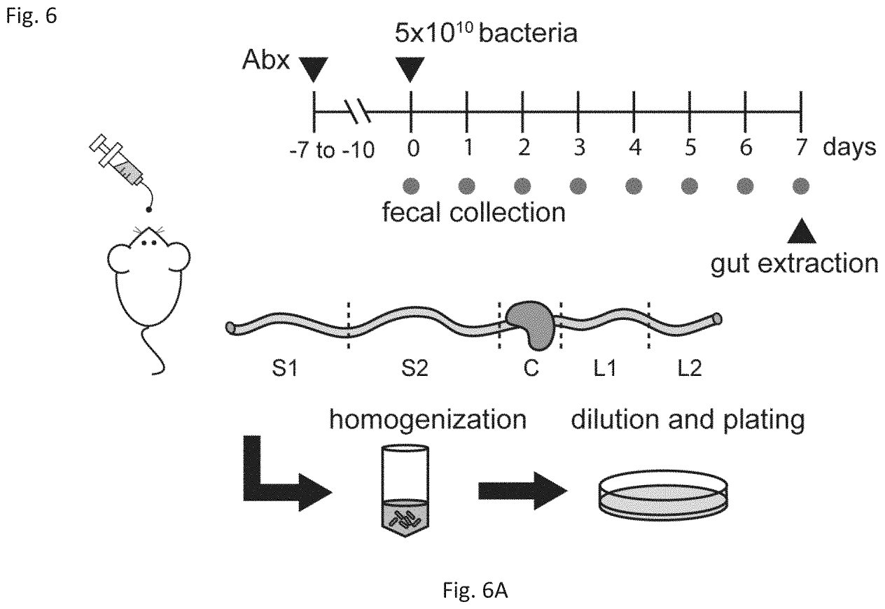

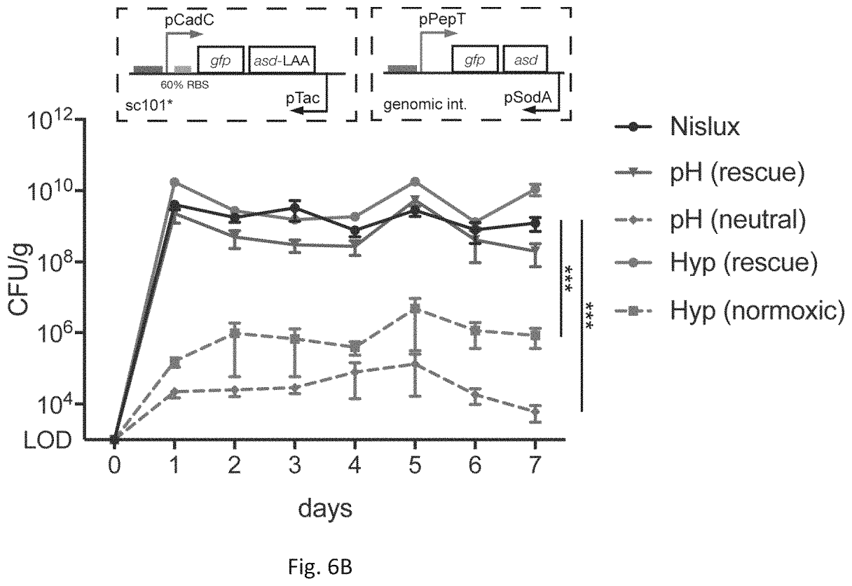

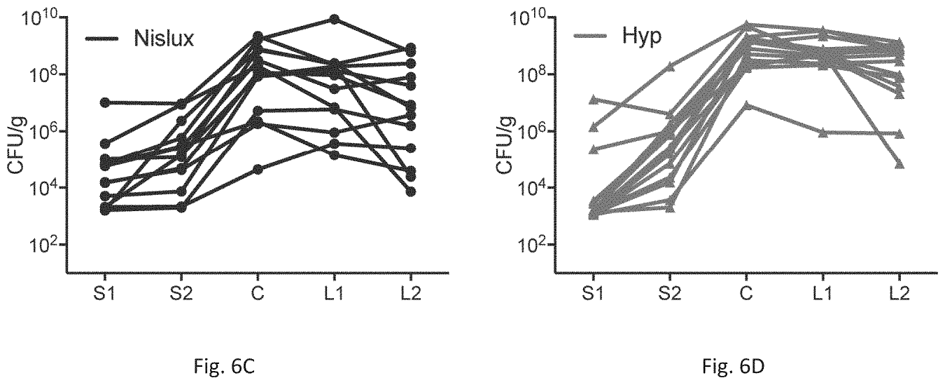

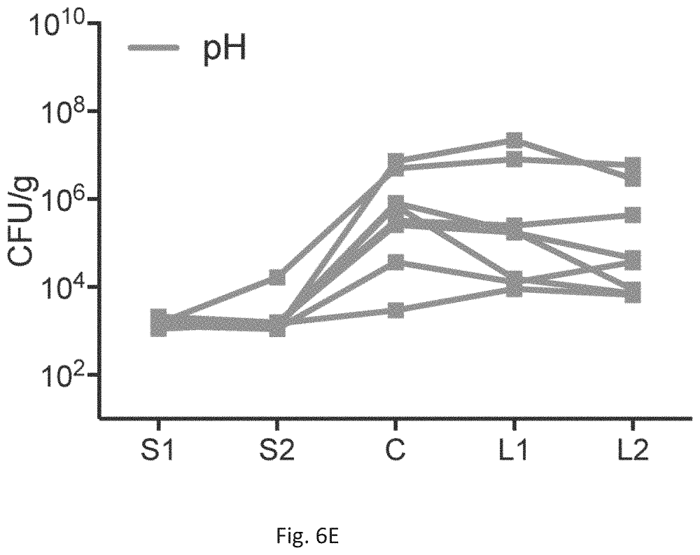

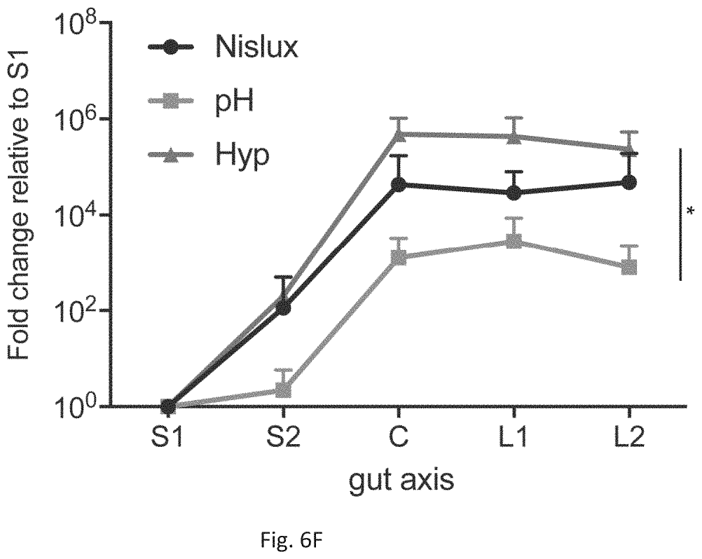

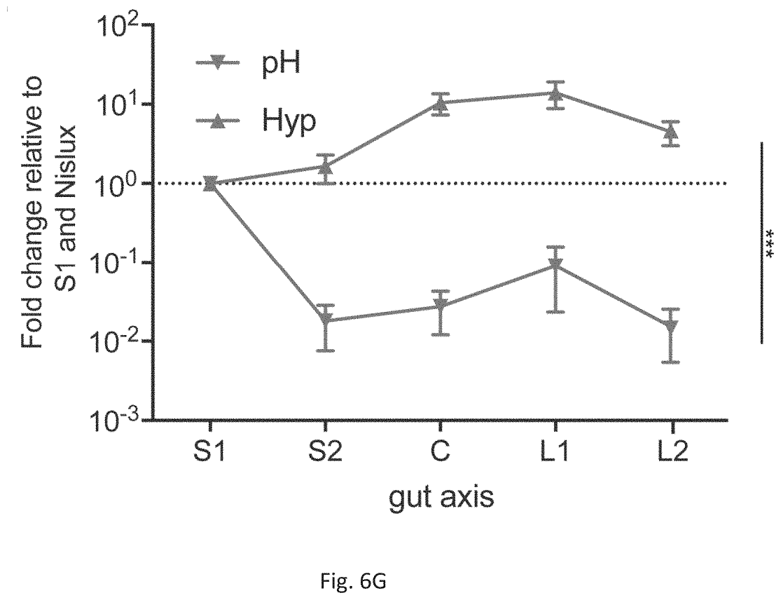

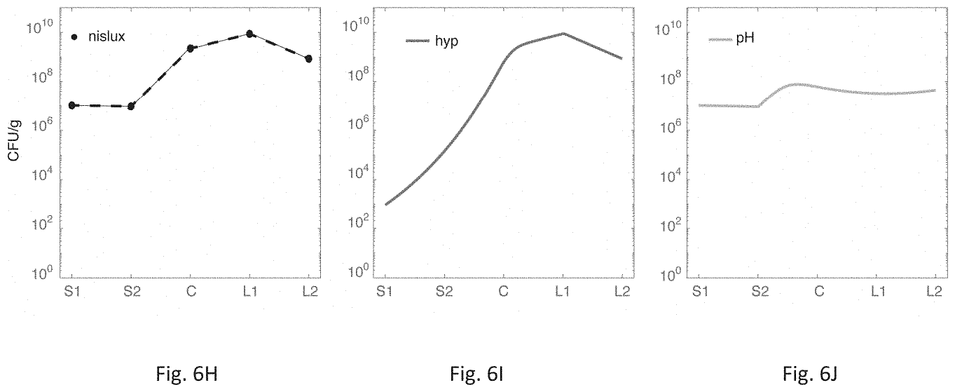

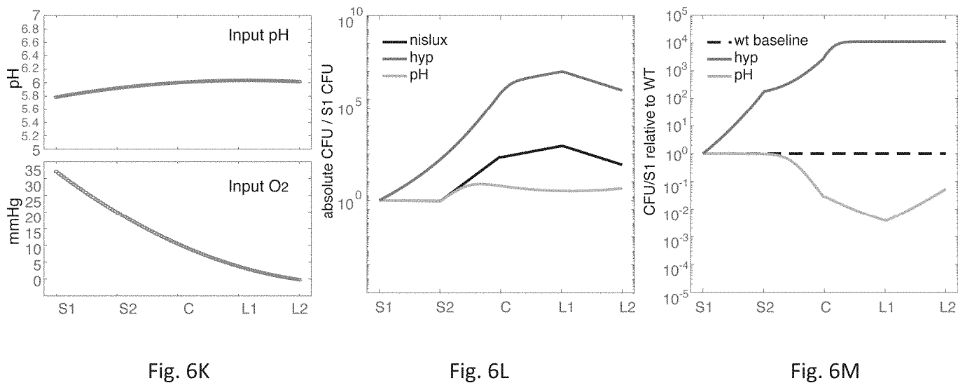

Measurements of gastrointestinal bacteria distribution. Recovered CFU were all normalized by weight of organ in grams (g) as done previously (Din et al. 2016; Danino et al. 2015). Absolute CFU g⁻¹ of individual mice were divided by respective CFU g⁻¹ from the S1 region to compute relative bacterial distribution along the gastrointestinal axis (FIGS. 6C-6F). To compare between engineered and wildtype strains, engineered bacteria (hypoxia and pH containment strain) CFU g⁻¹ relative to S1 was further normalized by wildtype bacteria from the 5 gut regions (FIGS. 6C-6F) for the purpose of describing bacterial distribution shifts.

Animal tumor models. All animal experiments were approved by the Institutional Animal Care and Use Committee (Columbia University, protocol AC-AAAN8002). The protocol requires animals to be euthanized when tumor burden reaches 2 cm in diameter or under veterinary staff recommendation. Mice were blindly randomized into various groups.

Animal experiments were performed on 6-8-week-old female BALB/c mice (Taconic Biosciences) with bilateral subcutaneous hind flank tumors from CT26 colorectal cells. The concentration for implantation of the tumor cells was 5×10⁷ cells per ml in RPMI medium (no phenol red). Cells were injected at a volume of 100 μl per flank, with each implant consisting of 5×10⁶ cells. Tumors were grown to an average of approximately 150 mm³ before bacterial injections. Tumor volume was quantified using calipers to measure the length, width, and height of each tumor (V=L×W×H).

Bacterial administration for in vivo experiments. Bacterial strains were grown overnight in LB media containing appropriate antibiotics. A 1:100 dilution into media with antibiotics was started the day of injection and grown until an OD of approximately 0.4. Bacteria were spun down and washed 3 times with sterile PBS before injection into mice. Oral gavage and intravenous injections of bacteria were performed at a concentration of 5×10¹⁰ and 5×10⁸ cells per ml in PBS respectively and administered at a total volume of 100 μl.

Biodistribution. After 2 days of bacterial injection, mice were sacrificed to harvest tumors, spleen and liver. Subsequently, organs were weighed and homogenized using a gentleMACS tissue dissociator (Miltenyi Biotec; C-tubes). Homogenates were serially diluted and plated on LB-agar plates at 37° C. overnight. Colonies were counted and computed as CFU g⁻¹ of tissue. Tumor targeting capability was calculated by comparing tumor:spleen and tumor:liver ratio of wildtype 1301 and engineered lactate hypoxia AND gate circuit strain. Limit of detection (LOD) were determined by lowest CFU detectable per dilution of sample.

Ex vivo organ measurements. pH was determined using pre-calibrated microprocessor-based pH meter with spear ending (Oakton Instruments). pH measurements were taken with gastrointestinal tract cut open and lumen exposed in order to measure pH level of lumen with pH meter in contact with the lumen surface. Tumor, liver and spleen were extracted, weighted, and homogenized using gentleMACS tissue dissociator (Miltenyi Biotec; C-tubes) in 5 ml PBS containing 15% glycerol. pH measurements were taken from homogenate with compensation for added PBS. A pH reading was taken immediately after the animals were euthanized to minimize influence of post-mortem time on pH. Samples were covered by probe tip and stable reading was acquired. The pH meter was washed with distilled water between measurements. Lactate assays were also performed on the extracted organs. Standard protocol using lactate colorimetric assay kit II (BioVision) was performed and dilution from added PBS were compensated by calculation.

Mathematical Model. To describe the behavior of biosensors, a system of ordinary differential equations was developed that describe the dynamic expression of GFP (y) in response to varying levels of environmental lactate (L; Eq.1), oxygen (O₂; Eq.2) and pH (H⁺; Eq.3), regulated by transcriptional factors LldR, FNR and CadC, respectively. Each biosensor's simplified equation was derived from mass-action equations describing the binding of transcription factors to DNA operators with regulation by lactate, oxygen and pH, based on previous approaches for describing promoter activity via repressor and activator occupancy (Anderson et al. 2006; Low et al. 1999). For simplicity it was assumed timescales for binding and transcription reactions to be significantly faster than translation (Cao et al. 2019; Mimee et al. 2015; Nakatsuji et al. 2018). Bacterial growth was modeled using the logistic equation with Nₘₐₓ as the maximum population size, γ is the degradation rate and μ is the growth rate (Eq.4). For containment circuits, it was assumed that protein concentrations produced from asd and glms genes follow GFP concentrations from the below biosensor equations. For the general AND gate containment circuit, it was assumed growth rate is proportional to the product of glms and asd expression (Eq.5) (FIG. 4D, 4E).

$$\frac{d y}{d t} = \frac{{α}_{y}}{1 + ( \frac{{K}_{1}}{1 + {K}_{L} L} )} - {γ}_{y} y \qquad ( 1 )$$

$$\frac{d y}{d t} = \frac{{α}_{y}}{1 + {K}_{2} ( 1 + {K}_{O 2} {O}_{2} )} - {γ}_{y} y \qquad ( 2 )$$

$$\frac{d y}{d t} = \frac{{α}_{y}}{( 1 + {K}_{3} ) {H}^{+} + {K}_{H}} - {γ}_{y} y \qquad ( 3 )$$

$$\frac{d N}{d t} = μ N ( 1 - \frac{N}{{N}_{max}} ) - γ N \qquad ( 4 )$$

$$μ = a * glms * asd \qquad ( 5 )$$

Model Parameter Values. Model parameters were chosen based on fitting experimental GFP biosensor data (FIGS. 4A-4C). GFP activation was seen with the increase of L (lactate concentration) and H⁺ (protons), and decrease of O₂ (oxygen level). Other parameter used include the following: α_{y} (production rate), 1750-3000; γ_{y} (degradation rate), 0.01-1; K₁ (lldR dimer binding affinity to plldR promoter), 200; K_{L} (Lactate binding affinity to lldR dimer), 200; K₂ (FNR dimer binding affinity to pPepT promoter), 0.5; K_{O2} (O₂ binding affinity to FNR dimer), 0.4; K₃ (CadC binding affinity to pCadC promoter), 0.7; K_{H} (H⁺ interaction with CadC transcription activator), 10⁻⁶; Nₘₐₓ(maximum population), 1000; γ (rate of bacterial degradation), 2.5; a (gene expression to growth rate proportionality constant), 1.3×10⁻⁷ for more than one essential gene, 4.6×10⁻⁴ for single essential gene. The parameter a was derived from experimental data of comparing supplement concentration to bacterial growth (FIG. 4D, 4E).

Statistical analysis. Statistical tests were performed either in GraphPad Prism 7.0 (Student's t-test and ANOVA) or Microsoft Excel. The details of the statistical tests are indicated in the respective figure legends. When data were approximately normally distributed, values were compared using either a Student's t-test, one-way ANOVA for single variable, or a two-way ANOVA for two variables with Bonferroni correction for multiple comparisons. Mice were randomized into different groups before experiments.

### Example 2—Development and Characterization of Engineered Biosensors

To construct bacterial bio sensors that can distinguish unique organ environments, oxygen, pH and lactate were chosen as common physiological indicators with significant prior quantitation in mice and humans (Table 1) (Andersen et al. 2013; Bakker et al. 2013; Carreau et al. 2011; DeSantis and Singer 2015).

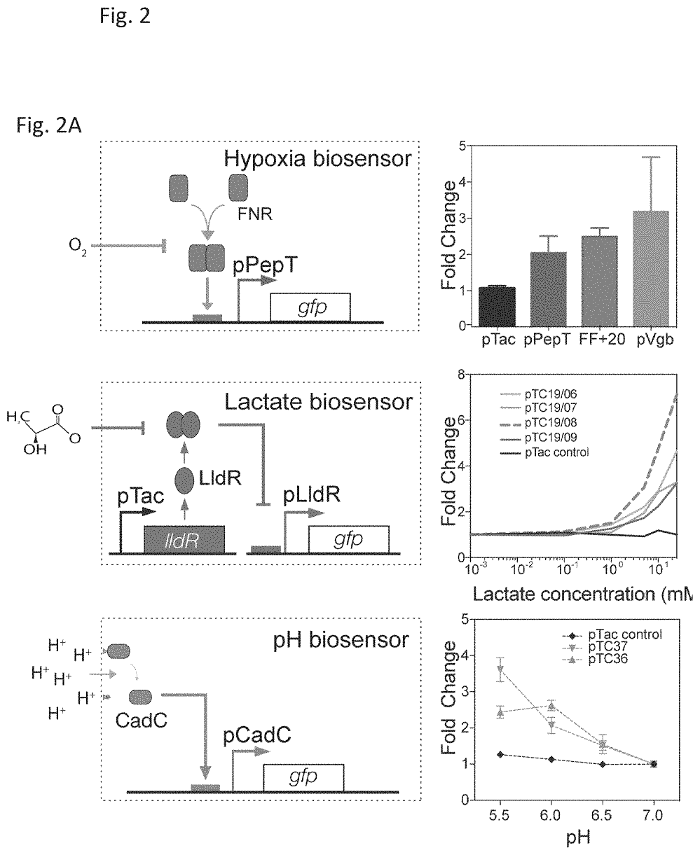

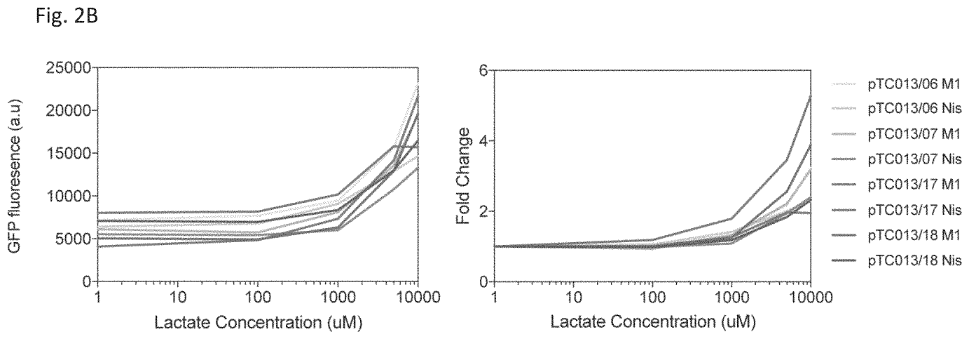

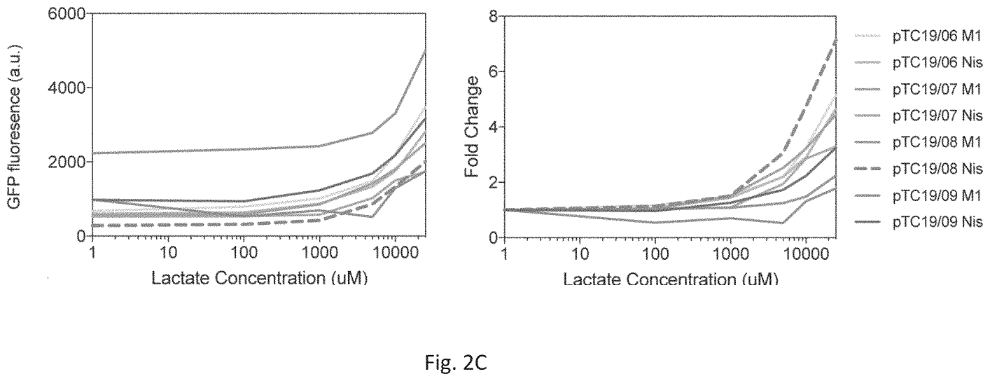

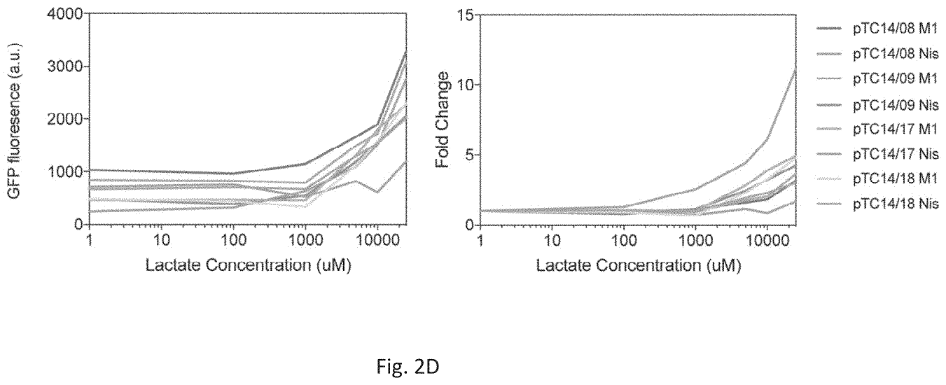

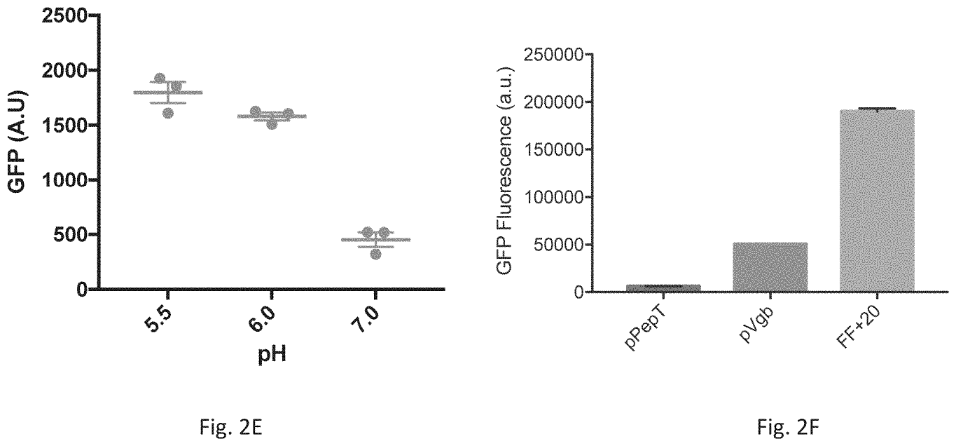

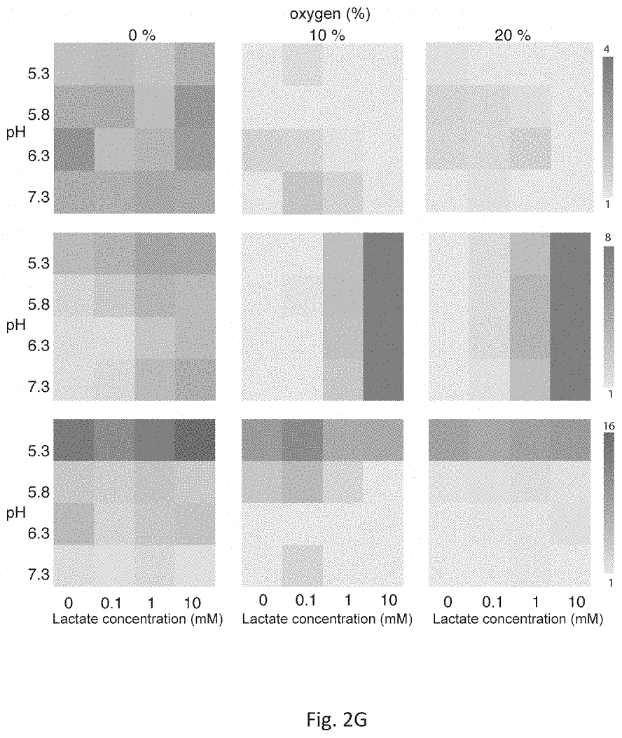

To sense oxygen, a hypoxia-sensing promoter (pPepT) was used that is primarily regulated by the transcriptional activator, fumarate and nitrate reduction regulatory protein (FNR) (Yu et al. 2012) (FIG. 2A). In the absence of oxygen, FNR binds a [4Fe-4S]²⁺ cluster to generate a transcriptionally active homodimer. However, the cluster is degraded in the presence of oxygen, which dissociates the FNR dimer into inactive monomers (Crack et al. 2008). Measuring GFP expressed under the control of the pPepT promoter on a plasmid, elevated levels of fluorescence were detected in response to hypoxic conditions (FIG. 2A).

Next, an L-lactate biosensor was designed, derived from the native LldPRD operon (Aguilera et al. 2008; Goers et al. 2017; Weghoff et al. 2015), to detect lactic acid fermentation by host mammalian cells. This lactate sensing system was constructed on two plasmids: a lactate-inducible reporter plasmid driving expression of a gene of interest and a repressor plasmid, which produces the repressor LldR that dimerizes to inhibit expression of the reporter gene unless bound to lactate (FIG. 2A). In response to increasing concentrations of L-lactate in the culture media, a corresponding increase in GFP was observed (FIG. 2A).

Lastly, a pH-sensitive promoter, pCadC was engineered, regulated by a membrane tethered activator protein (CadC) (Schlundt et al. 2017; Viala et al. 2011; Lee et al. 2008), which shows increased activity in acidic media compared to media at a neutral pH (FIG. 2A).

To establish biosensors that specifically activate in contrasting environmental conditions, a library of biosensor variants was built by tuning genetic parameters. For hypoxia biosensors, three distinct hypoxia promoters (pPepT, FF+20 and pVgb) were tested (Yu et al. 2012; Ryan et al. 2009; Mengesha et al. 2006) and demonstrated that hypoxia induced gene expression downstream of each promoter when compared to a constitutive promoter (FIG. 2A). For lactate biosensors, the origin of replication was varied as a knob to tune the copy numbers of the plasmids encoding the promoter (pLldR) and the regulatory protein (LldR). Utilizing this approach, several biosensor variants showed significant activation under increasing lactate concentrations with varied sensitivity and dynamic ranges (FIGS. 2A-2D). Lastly, for pH biosensors, a library of biosensor variants was also constructed by changing plasmid copy numbers and biosensor activation within the physiological pH range was observed (FIG. 2A, FIG. 2E).

A single biosensor variant for oxygen, lactate, and pH conditions was further characterized. The variants pPepT, pTC1908, and pTC37 were selected because of their activation in physiological concentrations similar to naturally-occurring levels in various organs, such as the gastrointestinal tract and solid tumors (Petrova et al. 2018; Donaldson et al. 2016). Here the hypoxia biosensor, pPepT, demonstrated low basal expression, which is suitable for preventing non-specific activation (FIG. 2F). The lactate biosensor variants generally demonstrated low basal expression, and pTC1908 was chosen additionally based on its relatively high dynamic range (FIGS. 2A-D). Specifically, pTC1908 is composed of a middle copy number reporter plasmid and its basal expression is quenched by LldR repressor on a high copy number plasmid. For the pH biosensor, pTC37 was chosen, engineered on a single low copy number plasmid since it exhibited robust sensing at lower pH ranges (FIG. 2E). This pH biosensor functions in buffered LB media, as there is observed biosensor activation in pH of 5.5-6.

Since bacteria are often subjected to multiple environmental conditions simultaneously in vivo, the cross-reactivity of the biosensors was tested in overlapping environmental conditions. Here, functional activity of biosensors exposed to multiple conditions was observed, suggesting the ability of using these biosensors in a combinatorial manner (FIG. 2G).

### Example 3—Engineering Biosensors as Containment Strains to Control Bacterial Growth

It was next sought to restrict bacterial replication in response to distinct biochemical signatures by expressing essential genes under the control of the biosensor promoters. This design allows for the coupling of biosensors with cellular replication to amplify the output. As a result, a large fold difference in bacterial number between environmental conditions can be achieved despite the relatively low dynamic range of each biosensor. An essential gene (asd) required in lysine, threonine, and methionine biosynthesis was chosen. Supplementation by diaminopimelic acid (DAP), a bacteria specific amino acid, allows for growth in asd-knockout strains (Yu et al. 2012). Importantly. DAP cannot be produced or metabolized from the host cellular environment, which provides an ideal strategy for biocontainment in vivo. The asd gene was knocked out from the genome of E. coli and the gene placed under the control of the three biosensor promoters. Since the initial circuits showed high levels of basal expression, this expression was reduced by constructing a library of variants with a range of gene copy numbers and introducing additional gene regulators (FIG. 3A). Briefly, the plasmid copy number was decreased to lower basal gene expression level (colE1, p15a, or sc101 plasmid origin of replications and a single genome integration). Additionally expression levels were tuned by introducing transcriptional (ribosomal binding site (RBS) strength and antisense RNA) and translational (protein degradation tag) controls (FIG. 3A). A complete listing of modifications is shown in Table 2.

To assess the impact of these modifications, circuit function were measured as the ratio of bacterial growth in non-permissive environments (normoxia, 0 mM lactate or neutral pH) to permissive conditions (0% oxygen, 10 mM lactate, or pH 6), which represents the “escapee rate” for characterizing the containment capability of the engineered bacteria, as has been used in other publications (Chan et al. 2016; Stirling et al. 2018; Gallagher et al. 2015). Several strain variants with low escapee rates were identified that could grow selectively under permissive conditions (FIG. 3B). For the hypoxia containment circuit (pTH6-1), the construct containing an additional gene (gfp) upstream of asd was genomically integrated, to reduce asd gene expression levels, followed by an antisense normoxia promoter, pSodA to reduce basal expression of asd. This optimized circuit design achieved selective bacterial growth in hypoxic conditions (FIG. 3B, Table 2). For the lactate containment circuit (pBK3-2/8), lowering RBS strength and building on the low copy plasmid were required for lactate dependent bacterial growth (FIG. 3B, Table 2). Lastly, for the pH containment circuit (pTC085), in addition to a reduced RBS strength, a degradation tag downstream of asd was included to lower basal gene expression (FIG. 3B, Table 2). The proportions of bacteria that grew in non-permissive conditions (escapee rate) were less than 10⁻⁴, le, 10⁻² for hypoxia, lactate, and pH containment circuits, respectively. The oxygen, lactate and pH levels at which these containment strains grew corresponded with the biosensor activation range (FIGS. 2A, 3C-3E). Taken together, engineered bacterial growth that is governed by three different environmental cues was demonstrated.

### Example 4—Multiplexing Biosensors to Improve Specificity

Since a single physiological condition can be similar in two organs, for example, hypoxia in both the large intestine and in tumors, integration of multiple signals can allow for further distinguishing of organ locations. Thus, to further enhance the specificity of the biocontainment circuits, an AND logic gate circuit was designed, which only permits bacterial replication in the presence of two different environmental conditions. To do this, an additional containment strain was engineered with the lactate biosensor driving expression of glms, an essential gene orthogonal to asd that encodes for a glucosamine-6-phosphate synthase that can be rescued with D-glucosamine (Kim et al. 2013). This engineered lactate containment circuit exhibited selective growth in the presence of 10 mM lactate with a 10⁻³ escapee rate (FIGS. 3F-3J). Next the glms-based lactate containment circuit was combined with the asd-based hypoxia containment circuit in a double knockout strain (FIG. 3K). This AND logic gate system grew when cultured under both low oxygen and high lactate, while we measured no colonies up to the limit of detection (LOD, 10² CFU/mL) for hypoxic- or lactate-only culturing conditions (FIGS. 3F and 3L). Importantly, the AND logic gate system demonstrated ˜10⁴ improvement of escapee rate (10⁸) compared to single biosensor containment circuits. These findings highlighted that using multiplexed biosensor-based logic gate circuit architecture can significantly improve growth specificity.

To further explore the functionality of single and multi-input biocontainment circuit architectures, a mathematical model to simulate bacteria growth regulated by one or more biosensor promoters was developed. To do this, the rate of reporter protein (GFP) production governed by different biosensor regulators (activators for hypoxia and pH biosensors, repressors for lactate biosensor) was described with a system of ordinary differential equations. Bacterial growth was simulated based on biosensor activation (FIGS. 4A-4C), and explored multiplexed environmental biocontainment using various biosensor combinations (FIGS. 4D-4F). It was observed that the relatively low dynamic range of the promoters was sufficient to drive bacterial growth and result in significant differences in bacterial number based on environmental inputs in the physiological ranges of interest (FIGS. 4D-4I). While some biosensors displayed slight differences in activation kinetics, the model predicted growth of bacteria in various specified environmental combinations over longer time-scales in in vitro experimental observations (FIGS. 4J-4L), and provided a generalizable model of circuit architectures to explore beyond the immediate system and applications described herein.

### Example 5—Biosensor Strains Sense Changes in Mammalian Cell Culture Environment

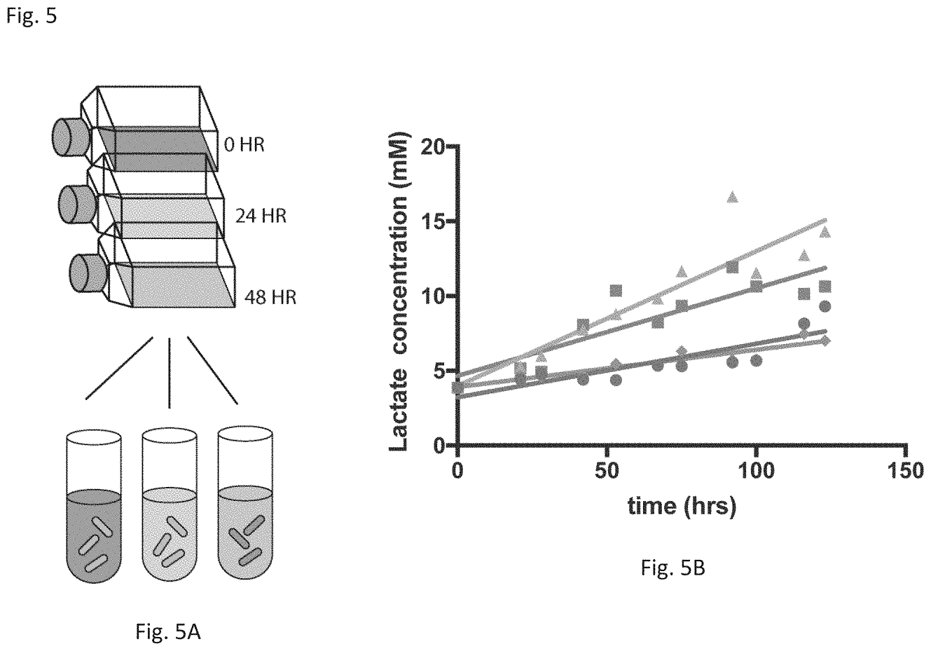

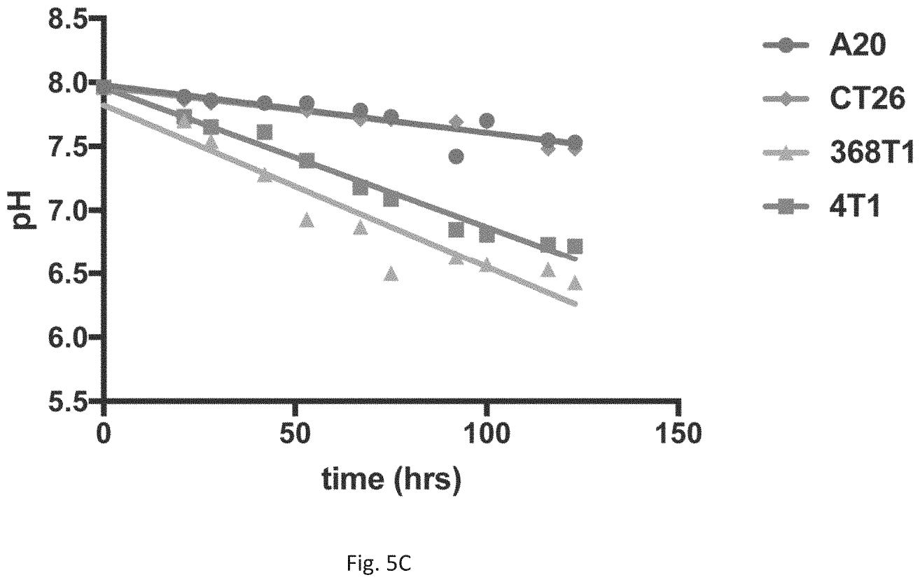

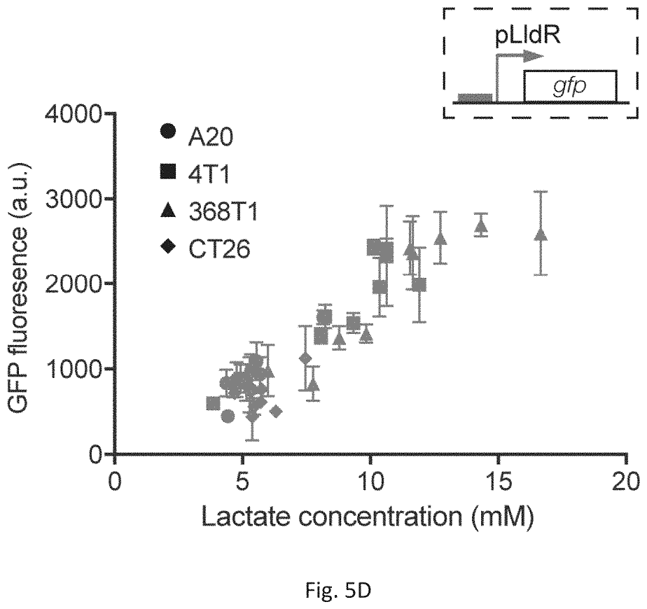

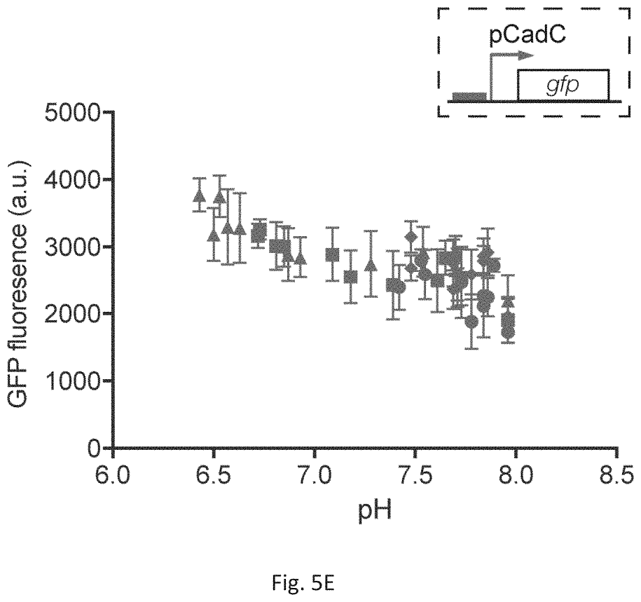

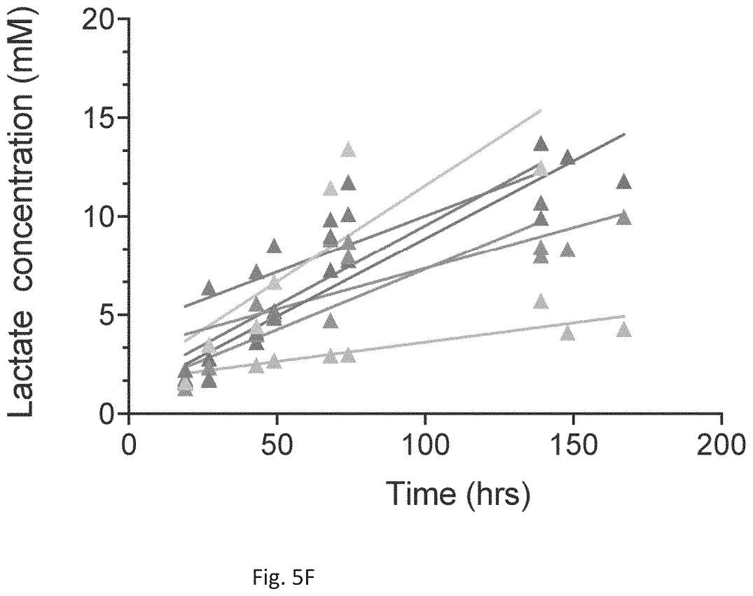

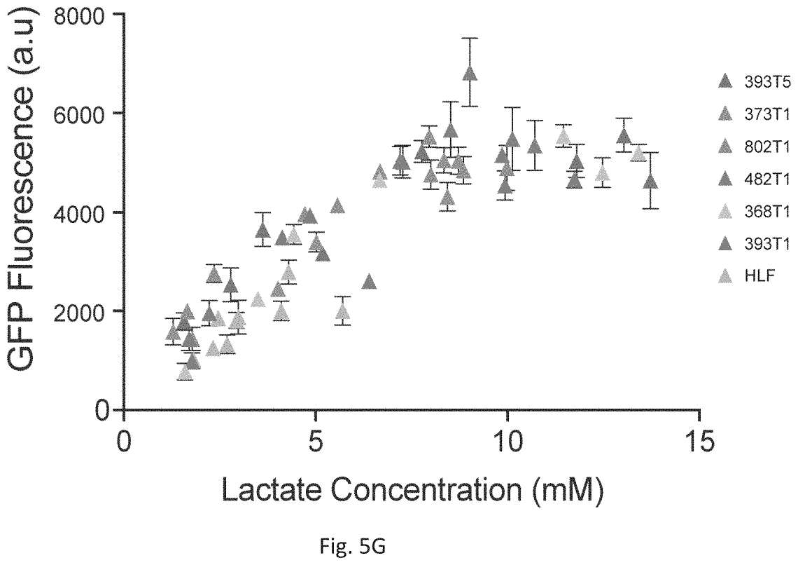

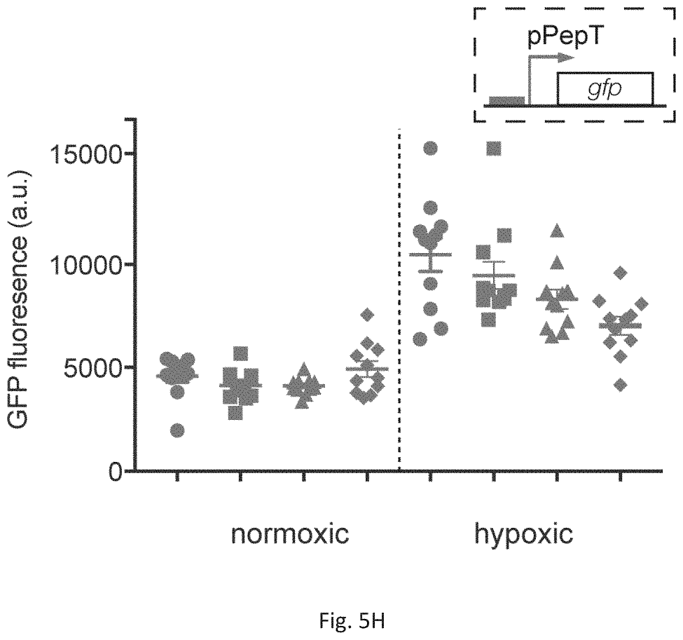

As a step towards in vivo characterization of biosensors, the capability of bacterial biosensors to sense metabolic activity of mammalian cell cultures in vitro was assessed first (FIG. 5A). Cell lines from various origins were cultured, including colorectal, lung, lymph nodes, and breast, over 5 days and measured the levels of lactate and pH in the culture media after collection (FIGS. 5B and 5C). Subsequently, biosensor-containing bacteria (pPepT, pTC1908, pTC37) was cultured in the collected cell media supernatant and measured the fluorescence from the biosensor strains after 16 hours. A concomitant increase in the bacterial fluorescent signal was observed as lactate concentrations increased and the pH level decreased (FIGS. 5D and 5E). To confirm that the biosensor activity is independent of cell origin, media supernatant samples from six different lung cancer cell lines and a lung fibroblast cell line were assayed (Winslow et al. 2011). It was found that the biosensor showed an increase in fluorescent signal only in the cancer cell lines that produced approximately 10 mM lactate, suggesting the activation range of our lactate biosensor to be within in vivo tumor levels (FIGS. 5F and 5G, Table 1). To characterize the hypoxia biosensor in culture media, the bacteria were incubated with cell media in a culture chamber with varying oxygen levels (0%, 10%, 20%). Consistent increases in fluorescence signal were observed following decreasing oxygen levels across cell lines (FIG. 5H). Collectively, these results demonstrated that the engineered bacteria can sense biochemical signatures produced from host cellular metabolic activities.

### Example 6—Containment Strains Shift Bacterial Distribution Along the Gut

Since oxygen and acidity levels decrease along the longitudinal axis of the intestine (Donaldson et al. 2016), this system was leveraged to engineer enhanced bacterial localization in the gut using the biosensor containment circuits. pH and oxygen biosensors driving asd gene expression were transformed into a probiotic bacterium, E. coli Nissle 1917, currently used for oral administration in humans with gastrointestinal disorders (Sonnenborn 2016; Wassenaar 2016). Following oral delivery of bacteria, fecal samples were collected over several days to assess their ability to grow outside of the host environment (FIG. 6A). For both hypoxia and pH containment strains, approximately 100-fold less bacteria from fecal samples grew during the course of one week compared to wildtype strain (labeled ‘Nislux’, E. coli Nissle 1917 with integrated luxCDABE cassette) (FIG. 6B). However, with DAP supplementation in the agar, containment strains were rescued to similar colony forming units (CFU) as in wildtype strain. These results suggested that the reduced bacterial numbers outside of the host occurred through engineered auxotrophy.

The bacterial distribution along the axis of the gut was subsequently examined by measuring CFU from homogenized intestinal tissue (FIG. 6A). Generally, more bacteria were found in the distal colon (caecum and large intestine) for all strains, as expected from previous studies (Donaldson et al. 2016; Sheth et al. 2019; Yasuda et al. 2015; Mark Welch et al. 2017 (FIG. 6C-6E). The pH containment strain showed lower levels of bacteria overall, potentially due to its reduced growth capability observed in vitro (FIG. 3E). To analyze relative bacterial distributions along the gut axis between strains, the gastrointestinal tract was compartmentalized into 5 regions and scaled to CFU/g recovered from S1 (FIG. 6F). Comparing to the wildtype strain, a 10-fold relative enrichment of hypoxia-dependent bacteria in the large intestine was found as compared to the small intestine (FIGS. 6C-6E, 6G). In contrast, a shift in bacterial distribution toward the proximal sites of the gastrointestinal tract for pH dependent bacteria was observed (FIGS. 6C-6E, 6G). Using the computational model, the shift in bacterial distribution was recapitulated along the gut axis with a corresponding input of oxygen and pH values (FIGS. 6H-6M, Table 1). Together, these results demonstrated enhanced bacterial tropism along the axis of the gastrointestinal tract based on physiochemical cues.

### Example 7—Combinatorial Hypoxia-Lactate Containment Strain Selectively Localizes to Tumors

Aside from modulation of bacterial distributions along the healthy gastrointestinal tract, it was next tested whether multiplexed containment circuits can enhance specificity in diseased sites such as tumors. In this context, bacteria such as S. typhimurium have been commonly studied as a cancer therapy due to their increased growth in tumor environments (Hohmann et al. 1996). While S. typhimurium has been reported to colonize tumors, it can also reach and survive in the liver and spleen (Yu et al. 2012; Zheng et al. 2017; Zhao et al. 2007; Hoffman 2016), suggesting the need to improve safety by reducing off-target bacterial load. It was sought to improve upon the natural tropism of S. typhimurium for tumors by integrating multiple unique metabolic signatures from the tumor microenvironment such as hypoxia, high lactate and low pH levels (Petrova et al. 2018; Chen et al. 2015; Ronero-Garcia et al. 2016; Sonveaux et al. 2008) (Table 1).

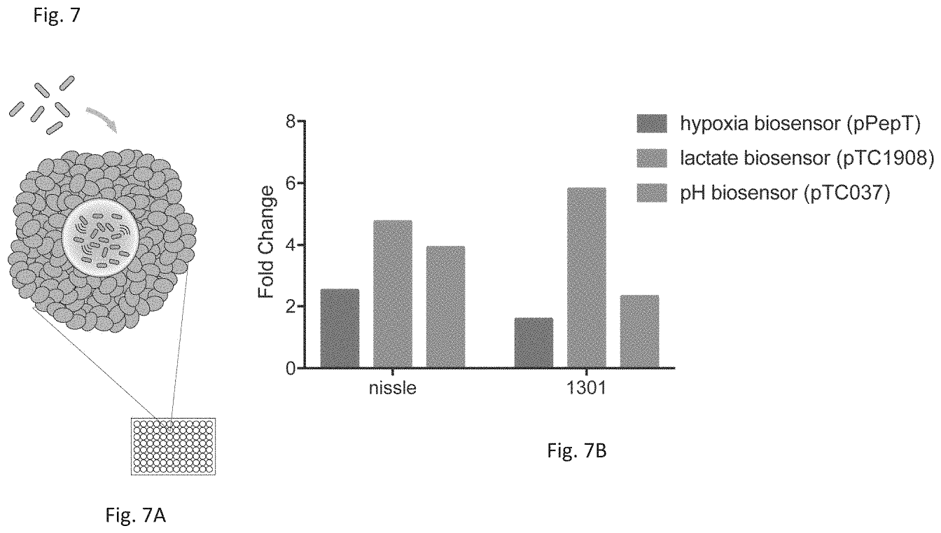

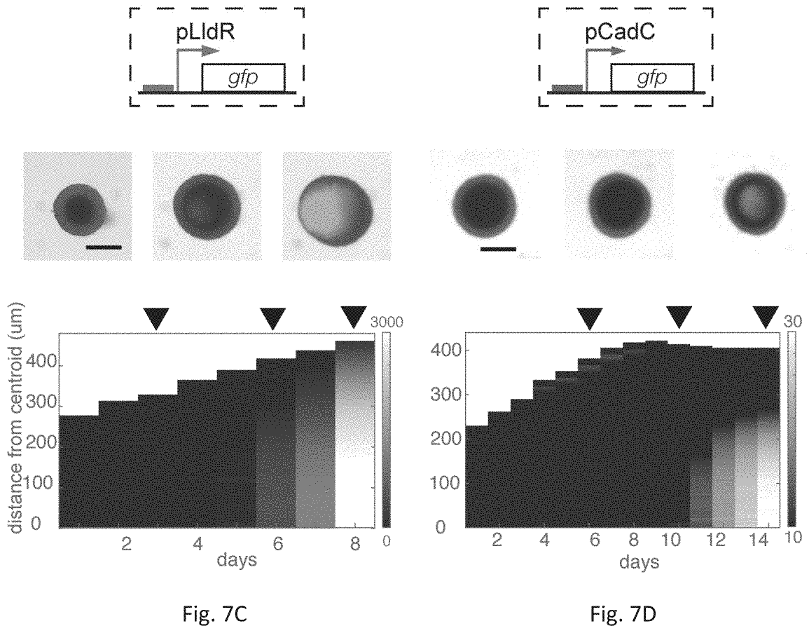

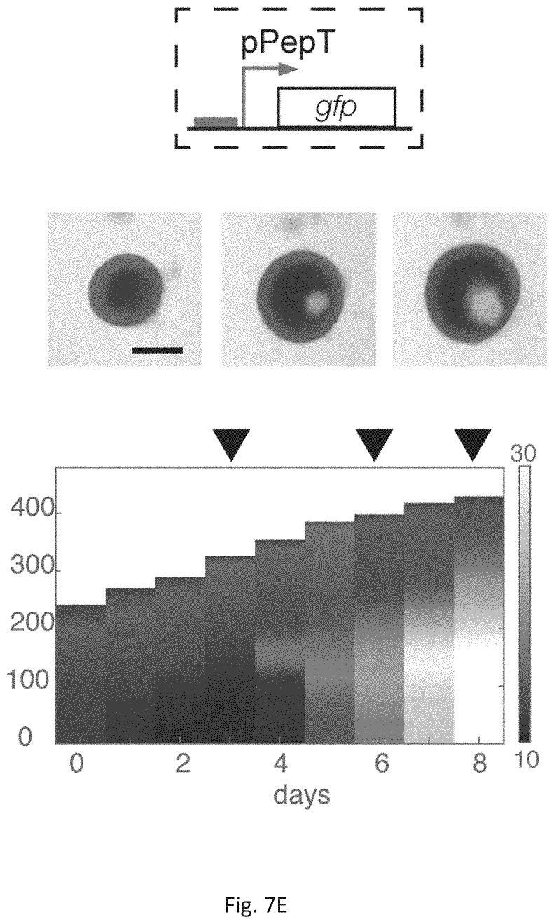

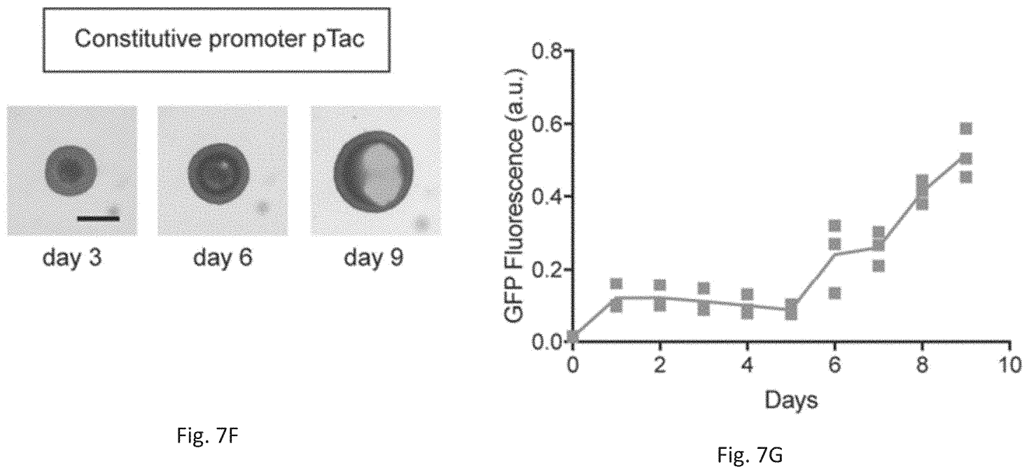

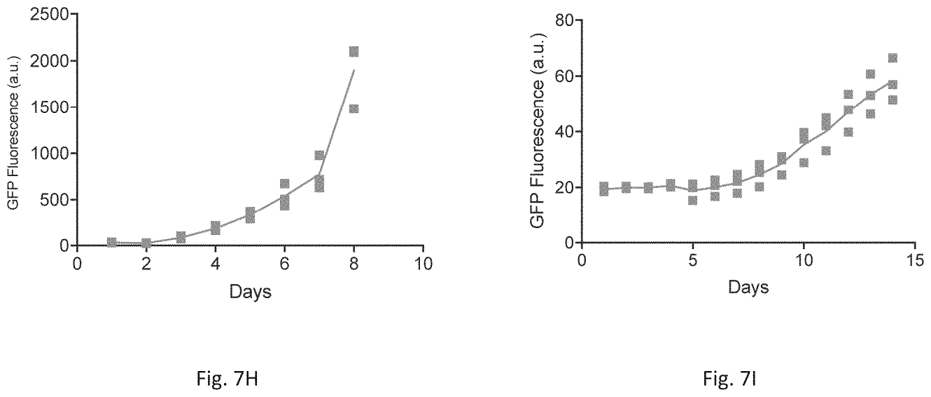

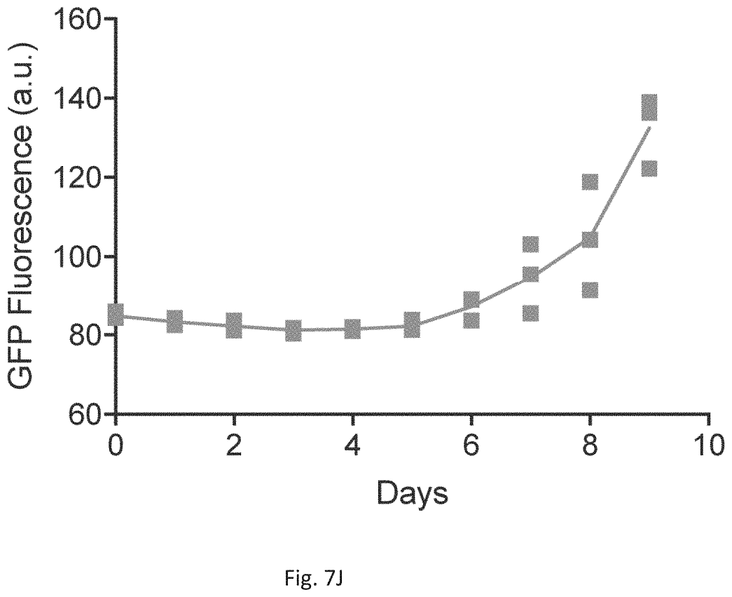

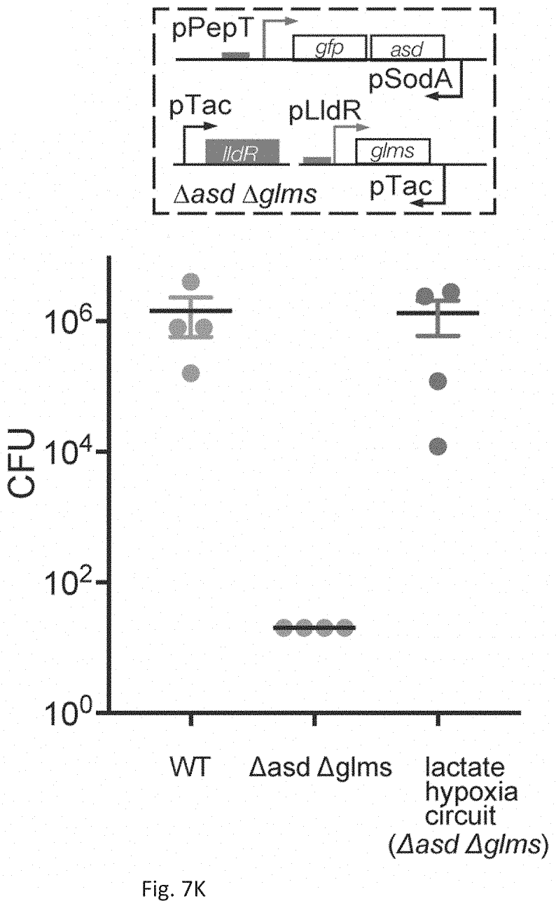

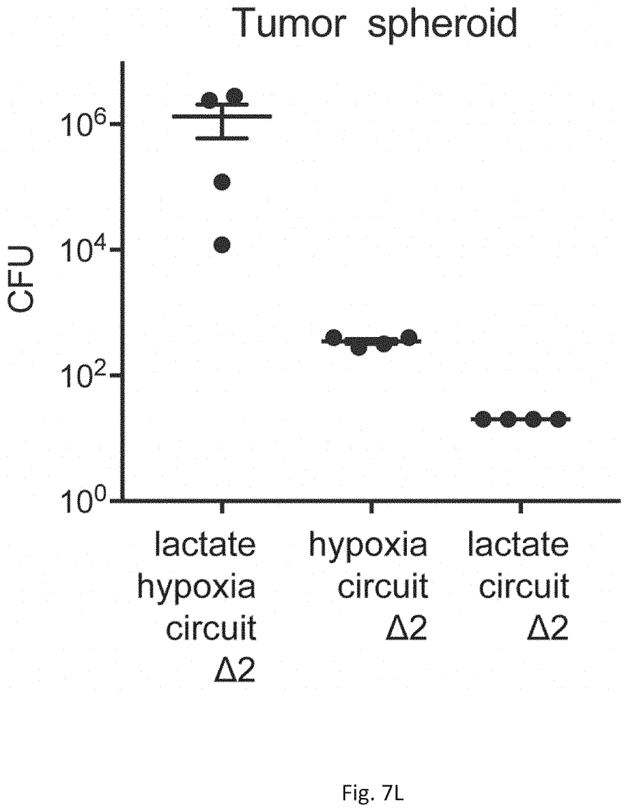

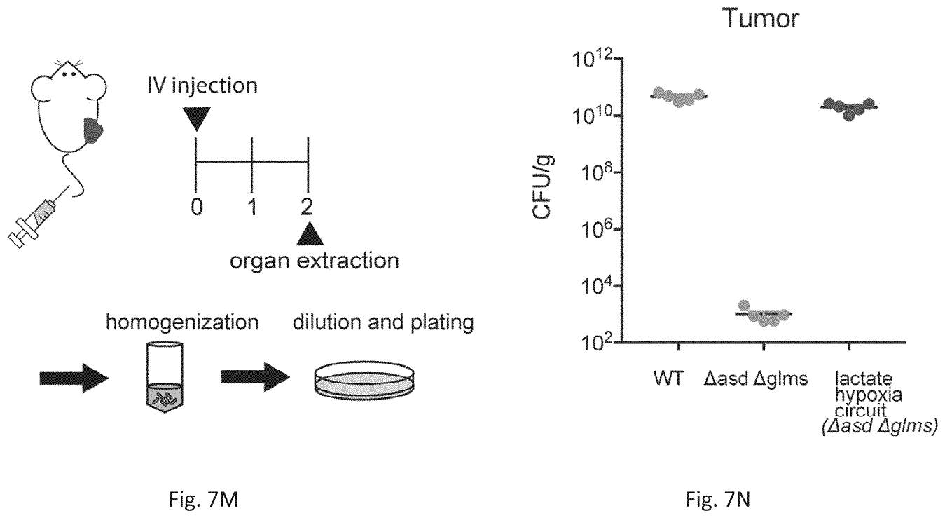

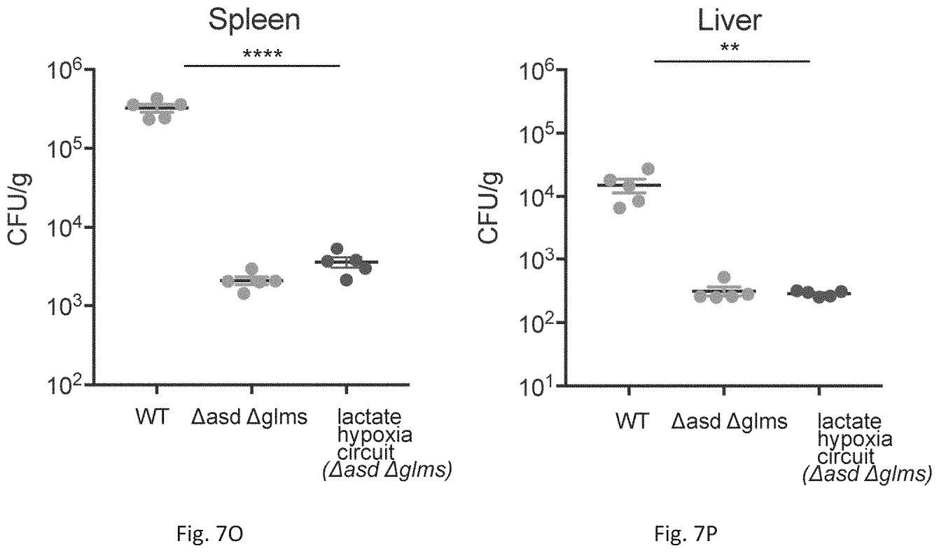

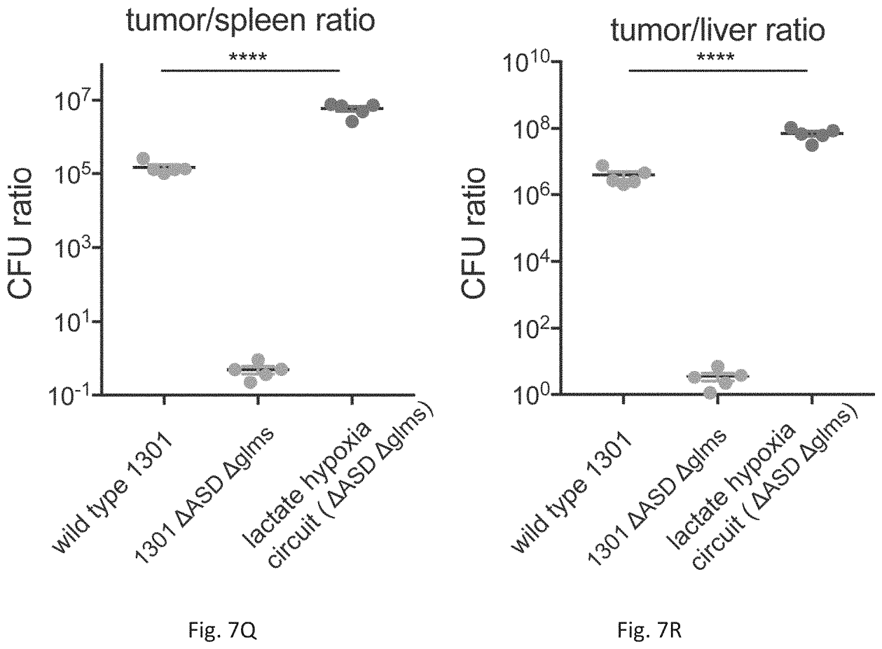

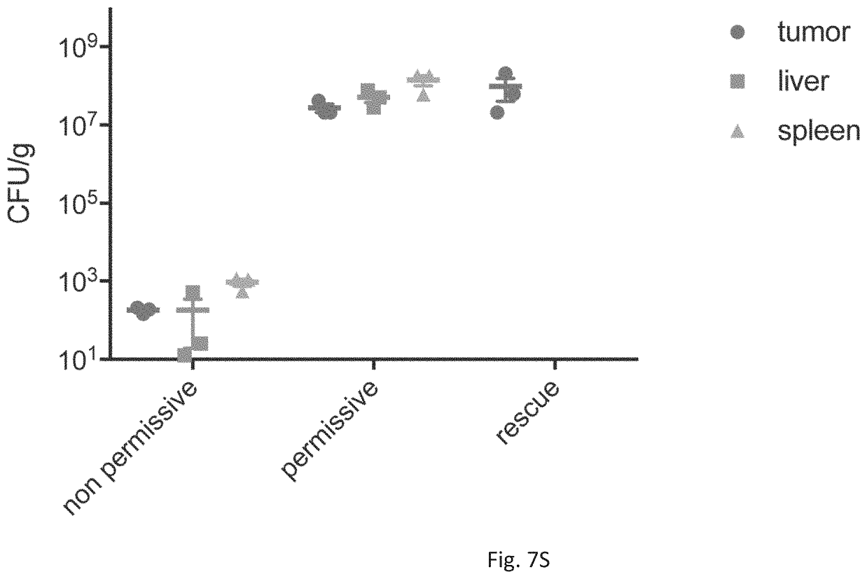

A recently designed three-dimensional bacteria spheroid co-culture system (BSCC) that recapitulates properties of the tumor microenvironment including oxygen and nutrient gradients, mammalian cell metabolism, and local 3D growth of the bacterial population in tumors was employed (Harimoto et al. 2019) (FIG. 7A). Attenuated S. typhimurium ELH1301 was transformed with biosensor plasmids (FIG. 7B) and co-cultured with the tumor spheroid system. Once in the spheroid core, an increase in the total fluorescence signal from bacteria carrying hypoxia, lactate, or pH biosensors driving GFP was observed (FIGS. 7C-7J). The highest reporter signals were consistently measured in the center of the spheroid, reflecting the expected biochemical gradients in the spheroid core (Harimoto et al. 2019; Walneta et al. 2000; Riffle et al. 2017). The combined lactate-hypoxia AND logic gate containment strain showed comparable tumor colonizing capabilities as wildtype bacteria (ELH1301) in tumor spheroids (FIG. 7K). To verify that the containment strain requires expression of both essential genes, strains where one biosensor drives a single essential gene in a double knockout strain (ELH1301 ΔasdΔglms) were constructed. Upon co-culture, these strains could not effectively colonize tumor spheroids (FIG. 7L), indicating that bacteria required both essential genes to grow.

The tumor containment strain (ELH1301 ΔasdΔglms with lactate-hypoxia AND gate) were intravenously injected in a subcutaneous syngeneic mouse tumor model (FIG. 7M). To measure improvement in tumor localization by the containment strain, a positive control (ELH1301 with no circuit) and negative control (ELH1301 ΔasdΔglms) strains that set the maximum and minimum bacterial load that can be expected from the organs upon systemic delivery, respectively were also administered. Organs and tumors were harvested and homogenized to assess bacterial colonization 2 days post-injection by CFU enumeration. As expected, lactate-hypoxia AND gate containment strain was observed to be able to colonize tumors to the similar level of the wildtype strain (FIG. 7N). These results suggested that the containment strain was able grow in the tumor environment that provides permissive lactate and oxygen levels. In comparison, the recovered CFU of the containment strain was lowered in the spleen and liver compared to 10⁴-10⁵ CFU/g bacterial load of the wildtype strain (FIGS. 7O and 7P). Since the double knock out (ΔasdΔglms) negative control strain was found to be at similar levels as the containment strain in the spleen and liver, it was reasoned that the non-tumor environment was unable to induce essential gene expression and permit growth. Based on these results, it was concluded that multiplexed bacteria decreased off-target colonization in the spleen and liver with a demonstrated greater than 100-fold increase in tumor-targeting specificity (FIGS. 7Q and 7R).

To test whether observed differences in bacterial growth were dependent on environmental signatures in situ, the engineered bacteria were grown in extracted organs ex vivo (tumor, liver, and spleen). No bacterial growth was observed in any homogenized organs that no longer possessed native physiological environments that were permissive for growth (20% oxygen, <1 mM lactate) (FIG. 7S). In contrast, significant increase in bacterial number were observed when these conditions were modulated for activation by spiking in 10 mM lactate and incubating at 0% oxygen conditions (FIG. 7S). Similar levels of bacterial growth were observed when rescued with essential supplements (DAP and D-glucosamine). These results indicated that the in vivo growth tropism of the engineered bacteria was independent of organ types and was primarily driven by the local hypoxia and lactate levels.

TABLE 1
Mouse physiological values from literature search and in vitro results relevant to the study
pH

| | Measured | |
|---|---|---|
| | (ex vivo, mean ± std) | Literature |
| Small intestine | S1: 5.76 ± 0.06 | SI: 4.8 (Stirling et al. 2018), ~7 |
| | S2: 5..95 ± 0.19 | (Chan et al. 2016) |
| Large intestine | C: 6.07 ± 0.13 | Caecum: 4.4 (Stirling et al. 2018), ~7 |
| | L1: 5.91 ± 0.01 | (Chan et al. 2016; Zhao et al. 2005) |
| | L2: 6.07 ± 0.26 | LI: 4.4-4.7 (Stirling et al. 2018) |
| Liver | 6.21 ± 0.18 | 7 (Piraner et al. 2017) |
| Spleen | 6.43 ± 0.04 | 7-7.6 (Yu et al. 2012) |
| Tumor | 6.24 ± 0.23 | 6.5 (Gallagher et al. 2015), |
| | | 6.7-6.8 (Huang et al. 2016), |
| | | 6.1-6.9 (DeSantis and Singer 2015), |
| | | 6-6.5 (Carreau et al. 2011), |

| | |
|---|---|
| Measured | |
| (ex vivo, mean ± std)* | Literature |

Lactate

| | | |
|---|---|---|
| | Measured | Literature |
| | (ex vivo, mean ± std)* | |
| Small intestine | SI1: 1.07 ± 0.30 μmol/g | SI: 30 nM (Andersen et al. 2013) |
| | SI2: 1.40 ± 0.25 μmol/g | |
| Large intestine | C: 1.00 ± 0.09 μmol/g | LI: 10 nM (Andersen et al. 2013), |
| | LI1: 1.11 ± 0.11 μmol/g | 2.5 mM (Aoi and Marunaka 2014), |
| | LI2: 1.26 ± 0.42 μmol/g | Caecum: 1 μmol/g (Zhao et al. 2005) |
| Liver | 5.16 ± 0.10 μmol/g | 1.95 μmol/g (Aoi and Marunaka 2014) |
| Spleen | 4.76 ± 0.08 μmol/g | 1.25 μmol/g (Crack et al. 2008) |
| Tumor | 9.47 ± 0.62 μmol/g | 14.3 μmol/g (Aoi and Marunaka 2014), |
| | | 8.14-17.8 μmol/g (Aguilera et al. 2008), |
| | | 7.3-25.9 μmol/g (Goers et al. 2017) |
| | | 10-40 mM (Weghoff et al. 2015; Schlundt et al. 2017) |

Oxygen

| | |
|---|---|
| Small intestine | Duodenum: 32 mmHg (Viala et al. 2011) |
| Large intestine | Ascending colon: 11 mmHg (Viala et al. 2011) |
| | Sigmoid colon: 3 mmHg (Viala et al. 2011) |
| | Caecum: <1 mmHg (1.3%) (Viala et al. 2011) |
| Liver | 44.39 ± 5.13 mmHg (Lee et al. 2008) |
| Spleen | 86 mmHg (Ryan et al. 2009) |
| Tumor | 8-10 mmHg (Schlundt et al. 2017; Mengesha et al. 2006) |

*μmol/g is equivalent to mM assuming 1 g of H₂O = 1 mL of H₂O

TABLE 2
Plasmids used in the study

| Identifier | Promoter | Relevant Features | ORI |
|---|---|---|---|
| pBK1 | pLldR | asd | sc101* |
| pBK2 | pLldR | asd-LAA | sc101* |
| pBK3 | pLldR | asd (AS) | sc101* |
| pBK3-1 | pLldR | 50% RBS asd (AS) | sc101* |
| pBK3-2 | pLldR | 10% RBS asd (AS) | sc101* |
| pBK6 | pLldR | GFP asd (AS) | sc101* |
| pTC06 | pTac | lldR | sc101* |
| pTC07 | pTac | lldRJldP | sc101* |
| pTC08 | pTac | lldR | colE1 |
| pTC09 | pTac | lldRJldP | colE1 |
| pTC13 | pLldR | GFP | colE1 |
| pTC14 | pLldR | GFP | sc101* |
| pTC17 | pTac | lldR | p15A |
| pTC18 | pTac | lldR, lldP | p15A |
| pTC19 | pLldR | GFP | p15A |
| pTC36 | pCadC | GFP | colE1 |
| pTC37 | pCadC | GFP | sc101* |
| pTC66 | pLldR | glms | sc101* |
| pTC77 | pCadC | asd-LAA | sc101* |
| pTC79 | pCadC | GFP asd | sc101* |
| pTC80 | pCadC | GFP asd (AS) | sc101* |
| pTC81 | pCadC | 11% RBS GFP asd-LAA | sc101* |
| | | (AS) | |
| pTC82 | pCadC | 29% RBS GFP asd-LAA | sc101* |
| | | (AS) | |
| pTC85 | pCadc | 60% RBS GFP asd-LAA | sc101* |
| | | (AS) | |
| pTH01-1 | pPepT | GFP | colE1 |
| pTH01-2 | pVgb | GFP | colE1 |
| pTH01-3 | Ff20 | GFP | colE1 |
| pTH07-1 | pPepT | asd | colE1 |
| pTH07-2 | pPepT | asd | p15A |
| pTH06-1 | pPepT | asd | Genome |
| | | | integrated |
| ptacsfgfp | pTac | GFP | colE1 |

TABLE 3
Bacterial and mammalian stains used in the study

| Label | Bacteria/Cell line | Genomic |
|---|---|---|
| EcN | Escherichia coli Nissle 1917 | N/A |
| EcN Δasd | Escherichia coli Nissle 1917 | Δasd |
| Keio collectionΔcadA | Escherichia coli | ΔcadA |
| Nislux | Escherichia coli Nissle 1917 | luxCDABE |
| | | (genomic) |
| Maeh1 | Escherichia coli | N/A |
| ELH1301 | S. typhimurium | N/A |
| ELH1301Δasd | S. typhimurium | Δasd |
| ELH1301Δglms | S. typhimurium | Δglms |
| ELH1301ΔasdΔglms | S. typhimurium | ΔasdΔglms |
| A20 | Mouse lymphoma cells | N/A |
| CT26 | Mouse colorectal carcinoma | N/A |
| CT26-1RFP | Mouse colorectal carcinoma | iRFP NLS |
| | | (genomic) |
| 4T1 | Mouse metastatic mammalian | N/A |
| | gland | |
| 368T1 | Mouse primary lung | N/A |
| | adenoc arcinomas | |
| 393T1 | Mouse primary lung | N/A |
| | adenoc arcinomas | |
| 373T1 | Mouse metastatic lung | N/A |
| | adenoc arcinomas | |
| 802T1 | Mouse primary lung | N/A |
| | adenoc arcinomas | |
| 482T1 | Mouse metastatic lung | N/A |
| | adenoc arcinomas | |
| 393T5 | Mouse metastatic lung | N/A |
| | adenoc arcinomas | |
| HLF | Human Lung Fibroblasts | N/A |

### REFERENCES

Aguilera et al., Dual role of LldR in regulation of the lldPRD operon, involved in L-lactate metabolism in Escherichia coli. J Bacteriol 190, 2997-3005 (2008).
Andersen et al., Etiology and therapeutic approach to elevated lactate levels. Mayo Clin Proc 88, 1127-1140 (2013).
Anderson et al., Environmentally controlled invasion of cancer cells by engineered bacteria. Journal of molecular biology 355, 619-627 (2006).
Aoi and Marunaka, Importance of pH homeostasis in metabolic health and diseases: crucial role of membrane proton transport. Biomed Res Int 2014, 598986 (2014).
Bakker et al., Clinical use of lactate monitoring in critically ill patients. Ann Intensive Care 3, 12 (2013).
Cao et al., Camouflaging bacteria by wrapping with cell membranes. Nat Commun 10, 3452 (2019).
Carreau et al., Why is the partial oxygen pressure of human tissues a crucial parameter? Small molecules and hypoxia. J Cell Mol Med 15, 1239-1253 (2011).
Chan et al., ‘Deadman’ and ‘Passcode’ microbial kill switches for bacterial containment. Nat Chem Biol 12, 82-86 (2016).
Chen et al., Evaluations of Tumor Acidosis Within In Vivo Tumor Models Using Parametric Maps Generated with Acido CEST MRI. Mol Imaging Biol 17, 488-496 (2015).
Chen et al., Tuning the dynamic range of bacterial promoters regulated by ligand-inducible transcription factors. Nat Commun 9, 64 (2018).
Crack et al., Signal perception by FNR: the role of the iron-sulfur cluster. Biochem Soc Trans 36, 1144-1148 (2008).
Daeffler et al., Engineering bacterial thiosulfate and tetrathionate sensors for detecting gut inflammation. Mol Syst Biol 13, 923 (2017).
Din et al., Synchronized cycles of bacterial lysis for in vivo delivery. Nature 536, 81-85 (2016).
Danino et al., Programmable probiotics for detection of cancer in urine. Science translational medicine 7, 289ra284-289ra284 (2015).
DeSantis and Singer, Tissue oxygen tension monitoring of organ perfusion: rationale, methodologies, and literature review. Br J Anaesth 115, 357-365 (2015).
Donaldson et al., Gut biogeography of the bacterial microbiota. Nat Rev Microbiol 14, 20-32 (2016).
Gallagher et al., Multilayered genetic safeguards limit growth of microorganisms to defined environments. Nucleic Acids Res 43, 1945-1954 (2015).
Goers et al., Whole-cell Escherichia coli lactate biosensor for monitoring mammalian cell cultures during biopharmaceutical production. Biotechnol Bioeng 114, 1290-1300 (2017).
Harimoto et al., Rapid screening of engineered microbial therapies in a 3D multicellular model. Proc Natl Acad Sci USA 116, 9002-9007 (2019).
Hoffman, Tumor-Targeting Salmonella typhimurium A1-R: An Overview. Methods Mol Biol 1409, 1-8 (2016).
Hohmann et al., Evaluation of a phoP/phoQ-deleted, aroA-deleted live oral Salmonella typhi vaccine strain in human volunteers. Vaccine 14, 19-24 (1996).
Hwang et al., Engineered probiotic Escherichia coli can eliminate and prevent Pseudomonas aeruginosa gut infection in animal models. Nat Commun 8, 15028 (2017).
Khalil and Collins, Synthetic biology: applications come of age. Nature Reviews Genetics 11, 367-379 (2010).
Kim et al., A novel balanced-lethal host-vector system based on glmS. PLoS One 8, e60511(2013).
Kitada, et al., Programming gene and engineered-cell therapies with synthetic biology. Science 359, (2018).
Lee et al., Next-generation biocontainment systems for engineered organisms. Nat Chem Biol 14, 530-537 (2018).
Lee et al., The membrane-bound transcriptional regulator CadC is activated by proteolytic cleavage in response to acid stress. Journal of Bacteriology 190, 5120-5126 (2008).
Low et al., Lipid A mutant Salmonella with suppressed virulence and TNFalpha induction retain tumor-targeting in vivo. Nat Biotechnol 17, 37-41 (1999).
Mandell et al., Biocontainment of genetically modified organisms by synthetic protein design. Nature 518, 55-60 (2015).
Mao et al., Probiotic strains detect and suppress cholera in mice. Sci Transl Med 10, (2018).
Mark Welch et al., Spatial organization of a model 15-member human gut microbiota established in gnotobiotic mice. Proc Natl Acad Sci USA 114, E9105-E9114 (2017).
Mengesha et al., Development of a flexible and potent hypoxia-inducible promoter for tumor-targeted gene expression in attenuated Salmonella. Cancer Biol Ther 5, 1120-1128 (2006).
Mimee, et al., Programming a Human Commensal Bacterium, Bacteroides thetaiotaomicron, to Sense and Respond to Stimuli in the Murine Gut Microbiota. Cell Syst 1, 62-71 (2015).
Nakatsuji et al., A commensal strain of Staphylococcus epidermidis protects against skin neoplasia. Sci Adv 4, eaao4502 (2018).
Petrova et al., The hypoxic tumour microenvironment. Oncogenesis 7, 10 (2018).
Piraner et al., Tunable thermal bioswitches for in vivo control of microbial therapeutics. Nat Chem Biol 13, 75-80 (2017).
Riffle et al., Linking hypoxia, DNA damage and proliferation in multicellular tumor spheroids. BMC Cancer 17, 338 (2017).
Riglar et al., Engineered bacteria can function in the mammalian gut long-term as live diagnostics of inflammation. Nat Biotechnol 35, 653-658 (2017).
Riglar and Silver, Engineering bacteria for diagnostic and therapeutic applications. Nature Reviews Microbiology 16, 214 (2018).
Romero-Garcia et al., Lactate Contribution to the Tumor Microenvironment: Mechanisms, Effects on Immune Cells and Therapeutic Relevance. Front Immunol 7, 52 (2016).
Ruder et al., Synthetic biology moving into the clinic. Science 333, 1248-1252 (2011).
Ryan et al., Bacterial delivery of a novel cytolysin to hypoxic areas of solid tumors. Gene Ther 16, 329-339 (2009).
Schlundt et al., Structure-function analysis of the DNA-binding domain of a transmembrane transcriptional activator. Sci Rep 7, 1051 (2017).
Sonnenborn, Escherichia coli strain Nissle 1917—from bench to bedside and back: history of a special Escherichia coli strain with probiotic properties. FEMS Microbiol Lett 363, (2016).
Sheth et al., Spatial metagenomic characterization of microbial biogeography in the gut. Nat Biotechnol 37, 877-883 (2019).
Sonveaux et al., Targeting lactate-fueled respiration selectively kills hypoxic tumor cells in mice. J Clin Invest 118, 3930-3942 (2008).
Stirling et al., Rational Design of Evolutionarily Stable Microbial Kill Switches. Mol Cell 72, 395 (2018).
Walenta et al., Metabolic imaging in multicellular spheroids of oncogene-transfected fibroblasts. J Histochem Cytochem 48, 509-522 (2000).
Wan et al., Cascaded amplifying circuits enable ultrasensitive cellular sensors for toxic metals. Nat Chem Biol 15, 540-548 (2019).
Wassenaar, Insights from 100 Years of Research with Probiotic E. Coli. Eur J Microbiol Immunol (Bp) 6, 147-161 (2016).
Weghoff, et al., A novel mode of lactate metabolism in strictly anaerobic bacteria. Environ Microbiol 17, 670-677 (2015).
Winslow et al., Suppression of lung adenocarcinoma progression by Nkx2-1. Nature 473, 101-104 (2011).
Viala et al., Sensing and adaptation to low pH mediated by inducible amino acid decarboxylases in Salmonella. PLoS One 6, e22397 (2011).
Yu et al., Explicit hypoxia targeting with tumor suppression by creating an “obligate” anaerobic Salmonella Typhimurium strain. Sci Rep 2, 436 (2012).
Yasuda et al., Biogeography of the intestinal mucosal and lumenal microbiome in the rhesus macaque. Cell Host Microbe 17, 385-391 (2015).
Zhao et al., Tumor-targeting bacterial therapy with amino acid auxotrophs of GFP-expressing Salmonella typhimurium. Proc Natl Acad Sci USA 102, 755-760 (2005).
Zhao et al., Monotherapy with a tumor-targeting mutant of Salmonella typhimurium cures orthotopic metastatic mouse models of human prostate cancer. Proc Natl Acad Sci USA 104, 10170-10174 (2007).
Zheng et al., Two-step enhanced cancer immunotherapy with engineered Salmonella typhimurium secreting heterologous flagellin. Sci Transl Med 9, (2017).

1. An engineered non-pathogenic bacteria comprising at least one essential gene under the control of an inducible promoter, wherein the non-pathogenic bacteria is deficient in the endogenous copy of the at least one essential gene.

2. The engineered non-pathogenic bacteria of claim 1, wherein the essential gene is chosen from the group consisting of asd, glms acpP, dxr, ipxC, hemA, nadE, ribA, folA, pyrH, adk, tmk, gmk and glnS.

3. The engineered non-pathogenic bacteria of claim 1, wherein the inducible promoter is chosen from the group consisting of promoters regulated by exogenous agents, promoters which are sensitive or respond to a particular environmental or physiological condition, and promoters which are induced by bacterial molecules.

4. The engineered non-pathogenic bacteria of claim 1, wherein the inducible promoter is chosen from the group of hypoxia-sensing, pH sensing, lactate sensing and combinations thereof.

5. The engineered non-pathogenic bacteria of claim 1, further comprising additional gene expression regulators chosen from the group consisting of antisense promoters, ribosome binding sites, and protein degradation tags.

6. The engineered non-pathogenic bacteria of claim 1, wherein the promoter is on a plasmid or integrated into the genome of the bacteria.

7. The engineered non-pathogenic bacteria of claim 6, wherein the plasmid is chosen from the group consisting of low copy, medium copy, and high copy.

8. The engineered non-pathogenic bacteria of claim 1, wherein the bacteria further comprises one or more plasmids encoding a nucleic acid which encodes a therapeutic agent and/or a diagnostic agent.

9. The engineered non-pathogenic bacteria of claim 1, wherein the bacteria comprises more than one essential gene under the control of an inducible promoter, wherein the more than one essential gene is under the control of inducible promoters sensing different physiological conditions.

10. The engineered non-pathogenic bacteria of claim 1, wherein the bacteria comprises more than one essential gene under the control of an inducible promoter, wherein the more than one essential gene is under the control of different inducible promoters and the inducible promoters are chosen from the group consisting of promoters regulated by exogenous agents, promoters which are sensitive or respond to a particular environmental or physiological condition, and promoters which are induced by bacterial molecules.

11. A composition comprising the engineered non-pathogenic bacteria of claim 2 and a pharmaceutically acceptable carrier.

12. (canceled)

13. A method of treating cancer in a subject in need thereof comprising administering to the subject a therapeutically effective amount of an engineered non-pathogenic bacteria comprising at least one essential gene chosen from the group consisting of asd, glms acpP, dxr, ipxC, hemA, nadE, ribA, folA, pyrH, adk, tmk, gmk and glnS under the control of an inducible promoter, wherein the non-pathogenic bacteria is deficient in the endogenous copy of the at least one essential gene.

14. The method of claim 13, wherein a therapeutically effective amount of more than one strain of engineered non-pathogenic bacteria is administered to the subject, wherein the more than one strain of bacteria comprise different inducible promoters.

15.-18. (canceled)

19. The method of claim 13, further comprising correlating the growth of the bacteria in a specific physiological condition to a location of cancer, a type of cancer and/or a genetic mutation of the cancer.

20. A method of treating a gastrointestinal disease or disorder in a subject in need thereof comprising administering to the subject a therapeutically effective amount of an engineered non-pathogenic bacteria comprising at least one essential gene chosen from the group consisting of asd, glms acpP, dxr, ipxC, hemA, nadE, ribA, folA, pyrH, adk, tmk, gmk and glnS under the control of an inducible promoter, wherein the non-pathogenic bacteria is deficient in the endogenous copy of the at least one essential gene.

21. The method of claim 20, wherein a therapeutically effective amount of more than one strain of engineered non-pathogenic bacteria is administered to the subject, wherein the more than one strain of bacteria comprise different inducible promoters.

22.-25. (canceled)

26. The method of claim 20, further comprising correlating the growth of the bacteria in a specific physiological condition to a location in the gastrointestinal system and/or a specific gastrointestinal disease or disorder.
